(19) United States
(12) Patent Application Publication
(10) Pub. No.: US 2002/0136785 A1
(43) Pub. Date: Sep. 26, 2002

(54) Ginseng berry extracts and pharmaceutical compositions from ginseng berry for the treatment of type 2 diabetes and obesity

(76) Inventor: Chun-Su Yuan, Chicago, IL (US)

Correspondence Address: MARK B WILSON, FULBRIGHT & JAWORSKI LLP, 600 CONGRESS AVENUE, SUITE 2400, AUSTIN, TX, 78701, US

(21) Appl. No.: 09/974,749

(22) Filed: Oct. 9, 2001

Related U.S. Application Data
(60) Provisional application No. 60/246,628, filed on Nov. 7, 2000.

(51) Int. Cl. A61K 35/78; A61K 31/704

(52) U.S. Cl. 424/728; 514/26

(57) ABSTRACT

The present invention relates to methods and compositions for the use in treating type 2 diabetes and obesity. More specifically, the invention relates to the methods of screening for the active compound from berries from plants of the Panax genus that decreases blood glucose and decreases body weight. It is contemplated that the active compound may comprise a ginsenoside or a combination thereof.

This application claims priority to U.S. Provisional Application No. 60/246,628, filed Nov. 7, 2000.

The government owns rights in the present invention pursuant to grant number DK31842 from the National Institutes of Health, grant number DK44840 from the National Institutes of Health and grant number P60DK2059 from the National Institutes of Health.

### BACKGROUND OF THE INVENTION

I. Field of the Invention

The present invention relates generally to the fields of physiology and medicine. More particularly, it relates to pharmaceutical compositions and the methods of screening for constituents that are anti-hyperglycemic or anti-obesity agents. Such constituents can be extracted from ginseng berry.

II. Description of the Related Art

A. Diabetes Mellitus

Diabetes mellitus is a major health problem, affecting approximately 5% of the total population in the U.S., and 3% of the population world-wide. Diabetes mellitus is a chronic metabolic disease that can cause blindness, kidney failure, or nerve damage. In addition, diabetes mellitus confers an increased risk of ischemic heart disease, stroke and peripheral vascular disease. Over 90% of diabetics are classified as type 2, or non-insulin-dependent diabetes mellitus (NIDDM); the rest fall into the category of type 1, or insulin-dependent diabetes mellitus (IDDM). Although the two types of diabetes have distinct pathogeneses, hyperglycemia and various life-threatening complications resulting from long-term hyperglycemia are the most common features.

1. Hyperglycemia in Type 2 Diabetes

Type 2 diabetes is characterized by fasting and post-prandial hyperglycemia. The fasting hyperglycemia is primarily caused by hepatic overproduction of glucose (Ferrannini et al., 1989; Kruszynska and Olefsky, 1996). Impaired suppression of hepatic glucose production, together with impaired glucose uptake by insulin-target tissues such as skeletal muscle is responsible for postprandial hyperglycemia (Firth et al., 1986; Mitrakou et al., 1990; Kruszynska and Olefsky, 1996). Chronic hyperglycemia is not only a marker of diabetes, but is also a factor which itself worsens metabolic control. By inhibiting both insulin secretion and glucose utilization, chronic hyperglycemia self-perpetuates the diabetic state (Yki-Jarvinen, 1992). Moreover, prolonged exposure to hyperglycemia causes production of oxygen free radicals which may lead to β-cell defects (Brownlee et al., 1984; Sakurai and Tsuchiya, 1988; Ihara et al., 1999).

Clinicians have suspected for many years that the complications of diabetes are secondary to chronic hyperglycemia. Now, a large body of data from epidemiological studies (Liu et al., 1993; Klein et al., 1994; Stolk et al., 1995) and clinical trials (Abraira et al., 1995; Ohkubo et al., 1995) strongly support the conclusion that hyperglycemia is the principal cause of retinopathy, nephropathy, neuropathy, and cardiovascular complications. These complications constitute the major clinical and economical burden of diabetes. Diabetic complications also significantly contribute to decreased quality of life. Available evidence indicates that sustained reductions in hyperglycemia will decrease the risk of developing microvascular complications, and most likely reduce the risk of macrovascular complications (Gaster and Hirsch, 1998). Despite evidence for the benefits of improved glycemic control, a large percentage of people with type 2 diabetes maintain poor glucose control, in part, because of the limitations of therapies for the management of diabetes (Klein and Klein, 1998).

2. Insulin Resistance in Type 2 Diabetes

One of the most important effects of insulin, with respect to type 2 diabetes, is the ability to stimulate glucose transport into tissues. Insulin-stimulated in vivo glucose disposal is markedly reduced in patients with type 2 diabetes (Shen et al., 1970; Ginsberg et al., 1975; Reaven, 1983). The main tissues that increase the rate of glucose uptake in response to an increase in plasma insulin levels are skeletal muscle and adipose tissue (Kruszynska and Olefsky, 1996). Skeletal muscle is the major site of glucose disposal (Defronzo et al., 1985), and consensus opinion suggests that in the majority of patients with type 2 diabetes, there is a defect in insulin-stimulated glucose disposal by skeletal muscle.

Many prospective studies of populations at high risk for type 2 diabetes (Lillioja et al., 1993; Taylor et al., 1994), and nondiabetic first degree relatives of patients with type 2 diabetes (Eriksson et al., 1989; Warram et al., 1990) have suggested that in most patients, the initial inherited lesion is insulin resistance. Thus, genetic factors are thought to have an important role in the development of diabetes. Recently, a putative diabetes gene was localized in the NIDDM1 region of chromosome 2. This putative diabetes-susceptibility gene encodes an ubiquitously expressed member of the calpain-like cysteine protease family, calpain 10 (CAPN10) (Horikawa et al., 2000).

Insulin resistance leads to a compensatory hyperinsulinemia, which is sufficient to maintain normal glucose tolerance, or at least impaired glucose tolerance. It is widely believed that with time, this compensatory mechanism becomes defective in a subset of patients due to β-cell failure, and leads to overt type 2 diabetes (Kruszvnska and Olefsky, 1996; Bailey, 1999).

Tissue glucose uptake is mediated by a family of five glucose transporters that have a tissue-specific distribution. One of them, GLUT4, is very responsive to an acute rise in insulin levels. GLUT4 is expressed only in skeletal muscle, adipocytes, and heart muscle (Shepherd et al., 1993). Insulin stimulates glucose transport by recruiting GLUT4-containing vesicles from the intracellular pool to the plasma membrane, and may also increase GLUT4 intrinsic activity (Shepherd et al., 1993; Guma et al., 1995; Kelley et al., 1996). The impairment of whole-body glucose utilization in type 2 diabetes is associated with defects in insulin-stimulated glucose transport in skeletal muscle (Bonadonna et al., 1993; Kelley et al., 1996), and adipocytes (Kashiwagi et al., 1983; Garvey et al., 1991).

Insulin resistance is independently associated with obesity, which accompanies 80% of patients with type 2 diabetes in the West (Kruszynska and Olefsky; 1996; Ferrannini, 1998). In addition, insulin resistance is more severe in obese patients with type 2 diabetes (Seely, 1993).

B. Ginseng

The ginseng root has been used for over 2000 years, in the belief that it is a panacea and promotes longevity. As described in Chinese traditional medicine textbooks, its effectiveness reaches mythical proportions (Lee, 1992; Huang, 1999). Seven major species of ginseng are distributed in East Asia, Central Asia, and North America (Huang, 1999). Most studies of ginseng have utilized constituents from common species including: Panax ginseng (Asian ginseng), Panax quinquefolius (American ginseng), and Panax japonicus (Japanese ginseng). However, the constituents from any of the species of ginseng currently known or discovered in the future would be expected to have utility.

Active constituents found in most ginseng species include ginsenosides, polysaccharides, peptides, polyacetylenic alcohols, and fatty acids (Lee, 1992). Ginsenosides are classified as steroidal saponins. There is a wide variation (2-20%) in the ginsenoside content of different species of ginseng (Huang, 1999). Moreover, pharmacological differences within a single species cultivated in two different locations have been reported. For example, the potency of extracts from Panax quinquefolius, cultivated in the U.S.A., for modulating neuronal activity is significantly higher than for the same species cultivated in China (Yuan, 1998b).

The dried root of Panax ginseng is a highly valued herb in the Far East, and has gained popularity in the West during the last decade. Previous investigations demonstrated that ginseng root possesses multiple pharmacological activities (Lee, 1992; Gillis, 1997; Yuan et al., 1998a, 1998b, 1999a; Huang, 1999). The two common species of ginseng are Panax ginseng (Asian ginseng), and Panax quinquefolius (American ginseng). To date, it is the root of ginseng that is well known as a herbal medicine. Commercially available ginseng extracts and powders are manufactured from the ginseng root, and previous studies have shown effects of the root (Kimura et al., 1981a; 1981b; Kimura and Suzuki 1981; Bensky and Gamble, 1993; Huang, 1999; Kimura et al., 1999).

1. In vitro Studies

Kimura (1980) reported that a partially-purified fraction of ginseng root enhanced glucose-stimulated insulin release from pancreatic islet cells of KK-CAy mice, which exhibits type 2 diabetes. In alloxan-diabetes mice, Kimura et al., (1981 a) showed that a water soluble fraction of ginseng root increased glucose-stimulated insulin release. Okuda and Yoshida (1980) reported that when adipose tissue of rats was incubated with an acidic peptide extracted from ginseng root, ACTH and growth hormone-induced lipolysis was inhibited, and lipogenesis was stimulated. Other studies have reported that an extract of ginseng root and the gensenoside Rb2 increased ATP levels in hepatic tissue of streptozotocin-induced diabetic rats (Yokozawa et al., 1991; Yokozawa and Oura, 1991). Ginsenoside Rb2 also increased albumin mRNA in the liver of streptozotocin-induced diabetic rats (Yokozawa et al., 1996). These studies did not provide any insight into the mechanisms of action of ginseng root.

2. In vivo Studies

Several studies have reported that a water extract of Panax ginseng root lowered fasting blood glucose levels in alloxan-induced diabetic mice (Kimura et al., 1981a; Kimura et al., 1999). In another study, Kimura et al., (1981b) showed that the glucose-lowering effect of a ginseng root extract fraction, in alloxan-induced diabetic mice, was abolished by antibodies to bovine insulin. In streptozotocin-induced diabetic rats, ginsenoside Rb2 caused a significant decrease in blood glucose levels, and regulated hepatic enzymes that maintain blood glucose within the physiological range (Yokozawa et al., 1985). Kimura and Suzuki (1981) reported that a fraction of ginseng root extract improved glucose tolerance in genetically diabetic KK-CAy mice. Except for the study by Kimura and Suzuki (1981), other in vivo studies are limited to the blood glucose-lowering effect of ginseng root extracts in type 1 diabetes models.

Other investigators have reported on the pharmacological effects of ginseng leaves in alloxan diabetic mice. Extracts of ginseng root and leaves increased basal plasma insulin levels and glucose-dependent insulin secretion, and prevented lipid peroxidation in the pancreas (Davydov et al., 1990). Molokovskii et al. (1989) reported that ginseng root and leaf extracts lowered blood glucose levels, and increased insulin and liver glycogen. Ginseng leaf extract was also reported to decrease the hypoglycemia caused by insulin infusion (Molokovskii and Barnaulov, 1986).

Two clinical trials that tested anti-diabetic effects of ginseng root have been reported. In a double-blind placebo-controlled study of ginseng root therapy for type 2 diabetes, Sotaneimi et al. (1995) reported that treatment with ginseng (species unknown) elevated mood, improved psychological performance, and reduced fasting blood glucose and body weight. A recent short-term clinical study by Vuksan et al. (2000) reported that American ginseng reduced post-prandial hyperglycemia in non-diabetic subjects, and in subjects with type 2 diabetes. The mechanisms of anti-hyperglycemic actions of ginseng have not been determined.

### SUMMARY OF THE INVENTION

The inventors describe herein pharmaceutical compositions comprising an active compound of ginseng berry extract and/or compounds found in ginseng berry that are useful as anti-hyperglycemic and anti-obesity agents. In certain embodiments of the invention, the inventors describe methods of screening for the active compound of ginseng berry extract. The present inventors envision that pharmaceutical compositions described herein may be able to modulate glucose homeostasis in an individual suffering from type 2 diabetes.

In specific embodiments of the present invention, pharmaceutical compositions are provideded comprising at least one active compound of from a berry from a plant of the Panax genus and a pharmaceutically acceptable carrier. The preferred species of ginseng in the present invention is Panax ginseng (Asian ginseng) or Panax quinquefolius (American ginseng). More specifically, the presently preferred Asian ginseng or Panax ginseng can be cultivated in Northeast China and the American ginseng or Panax quinquefolius can be cultivated in Wisconsin, U.S.A.

In certain embodiments, the active compound of the compositions comprises an anti-hyperglycemic constituent or an anti-obesity constituent or both an anti-hyperglycemic constituent and an anti-obesity constituent. More particularly, for example, the active compound comprises a ginisenoside. The single ginsenside may be Re. Other exemplary ginsenosides include, but are not limited to Rg1, Rb1, Rc, Rb2 or Rd. It is also contemplated that the active compound comprises at least two ginsenosides. Yet further, the active compound may comprise non-ginsenoside components of ginseng berry extract or may be ginsenoside free. One of skill in the art will recognize that the non-ginsenoside constituents may include, but are not limited to polysaccharides, peptides, polyacetylenic alcohols and fatty acids.

Another specific embodiment of the present invention provides a method of using the pharmaceutical compositions. The compositions may be administered to an animal suffering from non-insulin dependent diabetes, type 2 diabetes. The animal may be a mammal, such as a human. More specifically, in some embodiments, the human may be obese. Other exemplary mammals that can be treated using the present invention include, but are not limited to mice, rats, dogs, cats, guinea pigs, rabbits and monkeys.

In a further embodiment, the compositions may be administered via a parenteral route, such as, intraperitoneal, intravenous, subcutaneous, intramuscular, intradermal or transdermal.

In yet a further embodiment, the compositions may be administered via an alimentary route. Exemplary alimentary routes may include, but are not limited to oral, rectal, sublingual or buccal.

Another specific embodiment includes that the compositions may be administered as a dose. It will be understood that a dose can be the amount of the compositions administered to an animal necessary to achieve the desired effect during a given period of time. For example, the dose may be administered as a pill, an oral solution, an injectable solution or an infusable solution, e.g., a patch or a pump. It is contemplated that the dose can be administered at least once a day, but not excluding multiple doses, for example, two or more doses. Also, the dose may be administered pre-prandial. One of skill in the art will realize that the administration of an anti-hyperglycemic agent is governed by the fluctuations of blood glucose in an animal suffering from hyperglycemia. Because the levels of blood glucose can vary based upon a variety of situations, such as exercise, and diet, the administration of the pharmaceutical compositions may be altered to the needs of the animal to maintain glucose homeostasis.

In certain embodiments of the present invention, it is provided herein a method of using the pharmaceutical compositions as an anti-obesity agent. The compositions may be administered to an animal to increase body weight loss. It is contemplated that the increase in body weight loss may be caused by increases in energy expenditure and/or decreases in food intake.

Yet further, it is provided herein a method of using the pharmaceutical compositions as an anti-hyperglycemic agent. The compositions may be administered to an animal suffering from hyperglycemia. Also, the compositions may be administered to an animal to decrease blood glucose levels. It is contemplated that the decrease in blood glucose levels may comprise increases in tissue glucose uptake, which may be mediated by an increase in insulin sensitivity. Also, it is contemplated that the pharmaceutical compositions of the present invention may be administered in combination with a known anti-hyperglycemic agent. Thus, the present invention may be used to augment or enhance the function of the known anti-hyperglycemic agent. In addition to anti-hyperglycemic agents, the known agent may be an anti-diabetic agent or an anti-obesity agent.

In yet another specific embodiment, the present invention provides a method of using the pharmaceutical compositions to alter plasma cholesterol. For example, the compositions may be administered to an animal to decrease plasma cholesterol.

Another embodiment of the present invention provides a method of screening for an active compound from a berry from a plant of the Panax genus comprising: obtaining berry extract; and analyzing the extract for the active compound. Specifically, in some embodiments, the berry may be from the ginseng species Panax ginseng or Panax quinquefolius. After analyzing the extract for the active compound, the compound may be isolated, identified and synthsized using standard procedures that are well known to those in the art.

In specific embodiments, the extract may comprise at least one ginsenoside, for example, Rg1, Re, Rb1, Rc, Rb2 or Rd. Specifically, the ginsenoside is Re. Also contemplated is that the extract may comprise non-ginsenoside components or may be ginsenoside free.

In certain embodiments of the present invention, analyzing the berry extract may comprise separation of the extract into fractions by known mechanisms. More particularly, for example, separation of the berry extract may be accomplished by chromatography.

In yet a further specific embodiment, the method of screening for an active compound may further comprise administering the fractions obtained by chromatography separation to an animal suffering from hyperglycemia. After a fraction is administered to an animal, the method may further comprise measuring blood glucose, wherein a decrease in blood glucose indicates that the fraction contains an active compound. It is contemplated that the active compound may comprise a ginsenoside. Also, the active compound may comprise at least two ginsenosides. Yet further, it is contemplated that the active compound may comprise a non-ginsenoside component or may be ginsenoside free or may comprise a combination of a ginsenoside and a non-ginsenoside component.

Another specific embodiment of the present invention includes pharmaceutical compositions comprising an anti-hyperglycemic agent. The agent may comprise an active compound obtained by screening ginseng berry extract and admixing the agent with pharmaceutical compositions.

In certain embodiments of the present invention, a method of treating an animal suffering from non-insulin dependent diabetes is provided using an active compound from a Panax ginseng berry or from a Panax quinquefolius berry. The active compound may be isolated from Panax ginseng berry or a Panax quinquefolius berry extract. It is also contemplated that the active compound may be produced synthetically. Yet further, the active compound may be comprised in a pharmaceutically acceptable carrier.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the scope of the invention will become apparent to those skilled in the art from this detailed description.

### BRIEF DESCRIPTION OF THE DRAWINGS

FIG. 1: Chemical structures of ginsenoside Re, and Rb2 are shown.

FIG. 2: Percentage weight of six ginsenosides in fresh Panax ginseng berry vs. root using HPLC analysis.

FIG. 3A and FIG. 3B: Effect of Panax ginseng berry extract on fasting blood glucose concentrations in adult ob/ob mice (FIG. 3B) and lean littermates (FIG. 3A). On Day 0, glucose levels are higher in ob/ob mice compared to lean mice. Glucose levels decrease significantly in 150 mg/kg Panax ginseng berry extract-treated ob/ob mice on Day 5 and Day 12.

FIG. 4: Effect of Panax ginseng berry extract on fasting blood glucose levels in adult db/db mice. The glucose levels decreases significantly in 150 mg/kg extract-treated animals on Day 5 and Day 12.

FIG. 5: Effect of Panax ginseng berry extract on fasting blood glucose levels in adult lean littermates. Although there is a trend towards reduction in fasting blood glucose levels after the extract treatment, the glucose level does not decrease significantly on Day 12 compared to the vehicle-treated mice.

FIG. 6: Effects of Panax quinquefolius berry extract on blood glucose concentrations in ob/ob mice. 150 mg/kg=American ginseng berry extracts 150 mg/kg. In both vehicle group and treatment group, mean blood glucose levels on Day 0 were normalized to 100%.

FIG. 7: Dose-dependent effect of ginsenoside Re on fasting blood glucose concentrations in adult ob/ob mice. Compared to vehicle group, fasting glucose levels decreased significantly after 20 mg/kg ginsenoside Re treatments on Day 5 and Day 12.

FIG. 8: Effect of polysaccharides fraction from Panax quinquefolius berry extract on fasting blood glucose levels in adult ob/ob mice. The glucose levels decrease significantly in 50 mg/kg and 150 mg/kg polysaccharides-treated mice on Day 5 and Day 12.

FIG. 9: Prolonged hypoglycemic effect on fasting blood glucose levels in ob/ob mice treated with 150 and 50 mg/kg polysaccharides fraction from Panax quinquefolius berry extract. After 5 days (Day 15), 10 days (Day 20), and 15 days (Day 25) cessation of treatment, fasting blood glucose levels were significantly lower compared to the vehicle group.

FIG. 10A, FIG. 10B and FIG. 10C: Intraperitoneal glucose tolerance (IPGTT) test in ob/ob mice before and after a 12-day treatment with Panax ginseng berry extract. FIG. 10A shows vehicle-treated group. FIG. 10B shows 50 mg/kg extract-treated group. FIG. 10C shows 150 mg/kg extract-treated group, which has a significantly higher rate of glucose disposal at 30, 60 and 120 min.

FIG. 11A and FIG. 11B: IPGTT in adult db/db mice before (Day 0) and after a 12-day treatment with Panax ginseng berry extract or vehicle. (FIG. 11A) shows the 150 mg/kg extract-treated group, which has a significantly higher rate of glucose disposal at 30, 60 and 120 min. (FIG. 11B) shows the vehicle-treated group.

FIG. 12A and FIG. 12B: IPGTT in ob/ob mice before and after a 12-day treatment with Panax quinquefolius berry extract. FIG. 12A shows the vehicle treated group. FIG. 12B shows that mice treated for 12 days with 150 mg/kg of extract have a higher rate of glucose disposal at 60 and 120 min after 12 days of treatment vs. Day 0.

FIG. 13A, FIG. 13B and FIG. 13C. IPGTT in lean and ob/ob mice before and after a 12-day treatment with ginsenoside Re. FIG. 13A shows 20 mg/kg ginsenoside Re treatment in lean mice. FIG. 13B shows vehicle-treated ob/ob mice. FIG. 13C shows 20 mg/kg ginsenoside Re treatment in ob/ob mice, with a significantly higher rate of glucose disposal at 60 and 120 min compared to vehicle group.

FIG. 14A and FIG. 14B: IPGTT in ob/ob mice before (Day 0) and after a 10-day treatment with polysaccharides fraction from Panax quinquefolius berry extract. FIG. 14A shows 150 mg/kg polysaccharides-treated mice. FIG. 14B shows 50 mg/kg polysaccharides-treated mice. There is a significantly higher rate of glucose disposal based on measurement of area under the plasma concentration curve (AUC).

FIG. 15A and FIG. 15B: Effect of Panax ginseng berry extract on whole body glucose disposal during hyperinsulinemic-euglycemic clamps in ob/ob mice and lean littermates. Blood glucose levels (FIG. 15A) and steady state glucose infusion rates (FIG. 15B) obtained from the average rates of 120 min hyperinsulinemic euglycemic clamps were determined after 12-day treatment with 150 mg/kg extract.

FIG. 16A and FIG. 16B: Effects of Panax ginseng berry extract on body weight in ob/ob mice. FIG. 16A shows that there is a tendency to increase in body weight from Day 0 to Day 12 in mice received vehicle. 50 mg/kg extract ceases body weight increase. After 12 days of treatment with 150 mg/kg extract, body weight reduces significantly. FIG. 16B shows that after the cessation of 150 mg/kg extract treatment, ob/ob mice gradually regains body weight similar to vehicle-treated mice.

FIG. 17: Effects of Panax ginseng berry extract on body weight changes in adult db/db mice. Mean body weight on Day 0 is adjusted to 0%. There is a tendency to increase body weight from Day 0 to Day 12 in mice received vehicle. After 5 days and 12 days of 150 mg/kg extract treatment, body weight reduces significantly.

FIG. 18: Effects of Panax ginseng berry extract on body weight changes in adult lean littermates. Mean body weight on Day 0 is adjusted to 0%. After 5 days and 12 days of 150 mg/kg extract treatment, body weight reduces significantly.

FIG. 19: Effects of Panax quinquefolius berry extract on body weight in ob/ob mice. After 12 days of treatment with 150 mg/kg extract, body weight was reduced.

FIG. 20: Effects of polysaccharides fraction on body weight changes in ob/ob mice. While there is a tendency to increase in body weight from Day 0 to Day 10 in mice received vehicle, 150 mg/kg and 50 mg/kg polysaccharides treatment do not affect body weight changes.

FIG. 21: Effect of polysaccharides on body weight changes in ob/ob mice. Compared to vehicle group, 150 mg/kg and 50 mg/kg polysaccharides administration do not affect body weight changes during and after treatment.

FIG. 22: Effects of Panax ginseng berry extract on energy expenditure in ob/ob mice. After 12 days of treatment with 150 mg/kg extract, there was a significant increase in energy expenditure of the extract-treated group compared to the vehicle-treated group.

FIG. 23: Effects of Panax ginseng berry extract on energy expenditure in ob/ob mice. After 12 days of treatment with 150 mg/kg extract, there was a significant decrease in plasma cholesterol levels of the extract-treated group compared to the vehicle-treated group.

### DESCRIPTION OF THE ILLUSTRATIVE EMBODIMENTS

Currently, standard drug therapy for type 2 diabetes has a number of limitations, such as adverse effects and high rates of secondary failure. This has led to the search for alternative therapies that may have a similar degree of efficacy without the troublesome side effects associated with the conventional drug treatment. The inventors of the present invention envision that the identification of compounds from medicinal plants with anti-hyperglycemic and/or anti-obese activities may provide an opportunity to develop a novel class of anti-diabetic agents. Yet further, the compounds of the present invention may function as adjuvants in combination with other anti-diabetic agents.

Today, the number of patients with type 2 diabetes is growing in epidemic proportions throughout the world. Considering the heterogeneity of this disease, and the limitations of current therapies, such as high secondary failure rates, and side effects, there is an urgent need to explore new classes of anti-diabetic agents. In traditional Chinese and Japanese medical formulations, it is the root of Panax ginseng that was used for the management of diabetes. The present inventors demonstrate that other portions of the ginseng plant, such as the berry, also contain ginsenosides, but with a different chromatographic profile. Until the present invention, anti-hyperglycemic effects of ginseng berry have not been reported.

The active components of Panax ginseng are considered to be ginsenosides, a group of steroidal saponins (Huang, 1999; Attele et al., 1999). Ginsenosides are distributed in many parts of the ginseng plant, including the root, leaf and berry. The different parts of the plant contain distinct ginsenoside profiles (Huang, 1999), and these parts may have different pharmacological activities.

I Ginsenosides

Ginseng contains over twenty ginsenosides, and single ginsenosides have been shown to produce multiple effects in the same tissue (Tsang et al., 1985; Odashima et al, 1985). In addition, non-ginsenoside constituents of ginseng also exert pharmacological effects. Thus, one of skill in the art will realize that the overall activity of ginseng may potentially comprise a variety of pharmacological compounds.

A. Ginsenosides and Steroids

It is contemplated in the present invention that the active constituents may be a ginsenoside or a derivative thereof. For example, a single ginsenoside may be Re. Other examples of ginsenoside include, but are not limited to Rg1, Rb1, Rc, Rb2 or Rd. Ginsenosides (except Ro) belong to a family of steroids named steroidal saponins (Ota et al., 1987; Kim et al., 1998; Banthorpe, 1994). They have been named ginsenoside saponins, triterpenoid saponins, or damarene derivatives under previous classifications (Ourisson et al., 1964; Boar, 1983). Ginsenosides possess the four trans-ring rigid steroid skeleton, with a modified side chain at C-20 (Shibata et al., 1985). The classical steroid hormones have a truncated side chain (progesterone, cortisol, and aldosterone) or no side chain (estradiol and testosterone) (Banthorpe, 1994; Heftmann and Mosettig, 1960). Many steroids have a β-OH group at C-3; ginsenosides (for example, Rb1, Rb2, Rc, and Rd) usually have a sugar residue attached to the same site (Huang, 1999; Shibata et al., 1985). Sugar moieties are cleaved by acid hydrolysis during extraction, or by endogenous glycosidases to give the aglycone (Huang, 1999; Banthorpe, 1994; Shibata et al., 1985).

It is also contemplated that the ginsenoside of the present invention may function mechanistically similar to steroids. Thus, one of skill in the art will realize that the mechanisms of actions that apply to steroids may also apply to ginsenosides and is within the scope of the present invention. For example, steroids possess numerous physiological activities, partly due to the nature of the steroid skeleton, which is similar to a ginsenoside. The trans-ring junctions of the skeleton allow substituent groups, which interact with receptors, to be held in rigid stereochemically defined orientation (Banthorpe, 1994). In addition, the steroid skeleton endows the whole molecule with a favored structure to allow, for example, insertion into membranes (Bastiaanse et al., 1997). Recent work showed that Rg1 is a functional ligand of the nuclear glucocorticoid receptor (Lee et al., 1997; Chung et al., 1998).

B. Structural Diversity of Ginsenosides

Ginsenosides exhibit considerable structural variation. They differ from one another by the type of sugar moieties, their number, and their site of attachment. Some sugar moieties present are glucose, maltose, fructose, and saccharose. They are attached to C-3, C-6, or C-20. The binding site of the sugar has been shown to influence biological activity. Rh1 and Rh2 are structurally similar, except for the binding site of the β-d-glucopyranosyl group. In Rh1, the sugar is at C-6, and in Rh2, at C-3. Thus, it is contemplated in the present invention that the sugar moiety may be altered, e.g., substitutions of sugars or changes in position of the sugar, to potentially increase the pharmacological effect. These alterations are well known in the art and are within the scope of the present invention.

In another embodiment of the present invention, the site of a hydroxyl group may be altered to increase the efficacy of the constituent or to produce a different ginsenoside. Ginsenosides also differ in their number and site of attachment of hydroxyl groups. Polar substituents interact with phospholipid head groups in the hydrophilic domain of the membrane. Consequently, the insertional orientation of ginsenosides into membranes would be influenced by the number and site of polar OH groups. Differences in the number of OH groups were shown to influence pharmacological activity. Ginsenoside Rh2 and Rh3 differ only by the presence of an OH group at C-20 in Rh2.

Another factor that contributes to structural differences between ginsenosides is stereochemistry at C-20. Most ginsenosides that have been isolated are naturally present as enantiomeric mixtures (Banthorpe, 1994; Soldati and Sticher, 1980). Since the modules with which they react in biological systems are also optically active, stereoisomers are considered to be functionally different chemical compounds (Islam et al., 1997). Consequently, they often differ considerably in potency, pharmacological activity, and pharmacokinetic profile. Both 20(S) and 20(R) ginsenoside Rg2 inhibit acetylcholine-evoked secretion of catecholamines from cultured bovine adrenal chromaffin cells (Kudo et al., 1998). However, the 20(S) isomer showed a greater inhibitory effect. Thus, it is within the scope of the present invention that changes in stereochemisry may produce a different ginsenoside with a different potency. Such changes may be used to develop synthetically a constituent that has enhanced pharmacological activity that is non-naturally occurring in the berry extract.

Another embodiment of the present invention may include the use of other steroidal saponins to mimic the activity of ginsenoside. It is within in the scope of the present invention that other steroidal saponins may be modified to mimic and/or enhance the activity of the active ginsenoside constituent. It is also contemplated that these steroidal saponins or derivatives thereof may be synthetically produced for use in pharmaceutical compositions. One such example includes ganodermic acid S compounds, which are steroidal saponins that share structural features with ginsenosides (Shiao and Lin, 1987).

Also, another embodiment of the present invention includes determining the structural alterations in the ginsenosides in the gut after oral administration. It is believed that these alterations also contribute to diversity. Certain ginsenosides, such as Rb1 and Rg1, are poorly absorbed after ingestion (Odani et al., 1983). Rb1 was hydrolyzed to compound K by intestinal flora (Karikura et al., 1991); compound K was shown to increase the cytotoxicity of antineoplastic drugs (Hasegawa et al., 1995) and to induce apoptosis in B16-BL6 melanoma cells (Wakabayashi et al., 1998). Thus, it is also within in the scope of the present invention that these altered ginsenosides may be modified or encapsulated to prevent alteration after oral administration. Prevention of structural alteration may result in enhancement of the ginsenoside.

It is also within the scope of the present invention to alter other physiologic effects or pharmacodynamics of the ginsenosides. These alterations include enhancement of the effects of the ginsenosides, e.g., additive effects, synergism and potentiation. Thus, it is contemplated that there may be a synergistic effect and/or an additive effect between one or more than one ginsenoside or a ginsenoside(s) and another component(s) of the ginseng berry extract, e.g., polysaccharides or fatty acids. One of skill in the art will realize that synergism may occur to produce an effect that is greater in magnitude to the sum of the effects when the compounds (e.g., ginsenosides or other components of the ginseng berry) are given individually. Also, it will be realized that potentiation of compounds may occur. For example, but not limited to, a polysaccharide of the ginseng berry extract may not result in an anti-diabetic effect, however, in combination with a ginsenoside, the anti-diabetic effect may be enhanced beyond what is normal for the ginsenoside given alone.

II. Screening For the Active Compound of Ginseng Berry Extract

The present invention further comprises methods for identifying the active compound of ginseng berries. In specific embodiments, the ginseng berries are from the Panax ginseng (Asian ginseng) or Panax quinquefolius (American ginseng). These assays may comprise random screening of fruit extract for the candidate active compound or constituent. In particular, the assays may be used to focus on the particular ginsenoside moiety that is the active ginsenoside responsible for the anti-hyperglycemic effect and/or the anti-obesity effect. It is also contemplated that the active compound may comprise non-ginsenoside constituents or be ginsenoside free or essentially ginsenoside free or a combination thereof.

These candidate active compound constituents are assayed for the ability to modulate blood glucose, food intake, energy expenditure, plasma cholesterol, and/or body temperature.

To identify an active compound, one generally will prepare an extract of the fruit and analyze the extract. For example, a method generally comprises:

(a) obtaining berry extract; and

(b) analyzing the extract for the active compound.

Analyzing may comprise separation of the extract into fractions. These fractions may be assayed further to determine a difference between the measured characteristics of the fractions. A difference in the characteristics of the fractions, such as the ability to decrease blood glucose levels, indicate that the candidate fraction is, indeed, a constituent of the active compound.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Active Constituents

As used herein the term “candidate constituent or candidate substance” refers to any molecule that may potentially inhibit or enhance glucose utilization, body weight, food intake, cholesterol level, or any other metabolic parameter. It is contemplated that the candidate constituent is a ginsenoside or derivative thereof. The present invention is not intended to be limited to ginsenosides, other possible candidate constituents include, but are not limited to saponins, polysaccharides, cholesterol, peptides, polyacetylenic alcohols, fatty acids or other small molecules or derivatives thereof. It may prove to be the case that the most useful pharmacological compounds will be compounds that are structurally related to saponins. As used herein the term “saponin” refers to sapogenin glycosides, which is a type of glycoside widely distributed in plants. Saponins comprise a sapogenin, which constitutes the aglucon moiety of the molecule, and a sugar. It is contemplated that the sapogenin may be a steroid or a triterpane and the sugar moiety may be glucose, galatose, a pentose, or a methylpentose. Using lead compounds to help develop improved compounds is known as “rational drug design” and includes not only comparisons with known inhibitors and activators, but predictions relating to the structure of target molecules.

The goal of rational drug design is to produce structural analogs of biologically active target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound. In principle, this approach yields a pharmacore upon which subsequent drug design can be based. It is possible to bypass crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate molecules from banks of chemically- or biologically-produced molecules.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as plant sources, including leaves, bark, roots and fruit may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be saponins, cholesterol, other fatty acids or other small molecules or any other compounds that may be designed through rational drug design starting from known target compounds.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a candidate substance to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

Another example of an in vitro assay, may include incubating or bathing an isolated tissue, e.g., adipose or skeletal muscle, in a solution containing the candidate substance and measuring parameters that are well known in the art to change in response to insulin or glucose. Physiological and molecular analysis may be performed. For example, Northern or Western analysis may be preformed to measure changes in mRNA or protein levels.

C. In cyto Assays

The present invention also contemplates the screening of compounds for their ability to modulate metabolic activity in cells. Primary cell culture may be utilized to study various tissue responses in control and diabetic animal models. For example, hepatocyte primary cell cultures may be developed from livers of control and diabetic animals using standard techniques well known and used in the art of tissue culturing. Once the cell culture is established, these cells may then be incubated with various candidate substances and various parameters may be measured, e.g., biochemical or molecular. Biochemical assays may include, for example, enzyme analysis. Molecular analysis may be preformed, for example, looking at protein expression, mRNA expression and others.

D. In vivo Assays

In vivo assays involve the use of various animal models, including transgenic animals that have been engineered to have specific defects. In the present invention, the ob/ob mouse model is preferred. It is also contemplated that other diabetic transgenic mouse models may be used, for example, Jackson Laboratories has an extended inventory of transgenic diabetic mice, e.g., db/db mouse model. In addition to transgenic mice, chemically induced diabetic mouse and rat models are also available and they have also been contemplated and are within the scope of the present invention. These models include, but are not limited to, alloxan-diabetes mice and streptozotocin-induced diabetic rats.

Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons).

In specific embodiments, one or more candidate substances are administered to an animal, and the ability of the candidate substance(s) to alter one or more characteristics, as compared to a similar animal not treated with the candidate substance(s), identifies an active compound. The characteristics may be any of those discussed above with regard to the function alteration of blood glucose, plasma cholesterol, glucose utilization, energy consumption, food intake, body temperature and other metabolic parameter.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site. Also contemplated are transdermal routes, such as, via a patch.

Determining the effectiveness of a compound in vivo may involve a variety of different criteria. Also, measuring toxicity and dose response can be performed in animals in a more meaningful fashion than in in vitro or in cyto assays.

III. Chromatographic Separation Procedures

Any of a wide variety of chromatographic procedures may be employed according to the present invention. For example, thin layer chromatography (TLC), gas chromatography, high performance liquid chromatography (HPLC), paper chromatography, affinity chromatography or supercritical flow chromatography may be used to effect separation of various chemical species.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatography and thin-layer chromatography (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillar action draws the solvent through the paper, dissolves the sample, and moves the components in the direction of flow. Paper chromatograms may be developed for either ascending or descending solvent flow. Two dimensional separations are permitted by changing the axis of migration 90° after the first run.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In gas-liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long, as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High performance liquid chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

IV. Drug Formulations and Administration

Where clinical applications are contemplated, it will be necessary to prepare pharmaceutical compositions (e.g., active compounds, constituents of active compound, or an analog thereof) in a form appropriate for the intended application. Generally, this will entail preparing compositions that are essentially free of pyrogens, as well as other impurities that could be harmful to humans or animals.

Aqueous compositions of the present invention comprise an effective amount of the active compound or analog thereof, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances are well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, transdermal, subcutaneous, intramuscular, intraperitoneal or intravenous injection. Such compositions would normally be administered as pharmaceutically acceptable compositions, described supra.

The pharmaceutical forms of the active compound or analogs suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Prolonged absorption of the injectable compositions can be brought about by the use in the compositions of agents delaying absorption, for example, aluminum monostearate and gelatin.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

In a preferred embodiment for parenteral administration, the solution should be suitably buffered as necessary for the stability of the active compound or analog active ingredient and the liquid diluent first rendered isotonic with sufficient saline or glucose. Preferred pH range of the solution will be between 6.5 and 7.5. These particular aqueous solutions are especially suitable for intraperitoneal administration. In this connection, sterile aqueous media which can be employed will be known to those of skill in the art in light of the present disclosure.

The preferred dosage of the active compound in a parenteral administration may vary, depending upon the extent of the hyperglycemia, the severity of the symptoms associated with the hyperglycemia and patient age, weight and medical history. The number of administrations of the parenteral compositions of the active compound will also vary according to the response of the individual patient to the treatment. Further studies with animal models will completely define projected doses.

In other preferred embodiments of the invention, pharmacologically active compositions could be introduced to the patient through transdermal delivery of a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture.

Transdermal administration of the present invention may comprise the use of a “patch”. For example, the patch may supply one or more active substances at a predetermined rate and in a continuous manner over a fixed period of time.

Another preferred method of administering pharmacologically active compositions of active compound is as an aerosol. Aerosol compositions may be especially useful for the treatment of living tissue, although they could also be used for dermal applications. The term aerosol refers to a colloidal system of finely divided solid of liquid particles dispersed in a liquefied or pressurized gas propellant. The typical aerosol of the present invention for oral or nasal inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to patient age, weight and the severity and response of the symptoms.

For oral administration the active compound of the present invention may be incorporated with excipients and used in the form of non-ingestible mouthwashes and dentifrices. A mouthwash may be prepared incorporating the active compound in the required amount in an appropriate solvent, such as a sodium borate solution (Dobell's Solution). Alternatively, the active complex may be incorporated into an antiseptic wash containing sodium borate, glycerin and potassium bicarbonate. The active compound may also be dispersed in dentifrices, including: gels, pastes, powders and slurries. The active compound may be added in a therapeutically effective amount to a paste dentifrice that may include water, binders, abrasives, flavoring agents, foaming agents, and humectants.

V. Combined Therapy

In another embodiment, it is envisioned to use a candidate active compound in combination with other anti-diabetic or anti-obesity agents. These agents may include traditional agents, however, it may also include nontraditional compounds. Examples of traditional anti-diabetic agents include, but are not limited to, buformin, metformin, phenformin, insulin, acetohexamide, 1-butyl-3-mtanilylrea, carbutamide, chlorpropamid, glibornuride, gliclazide, flimeperidie, glipzide, gliquidone, glisoxepid, glyburide, glybuthiazol, glybuzole, glyhexamide, glymidine, glypinamid, phenbutamide, tolazamide, tolbutamide, tolcyclamide, pioglitazone, troglitazone, acarbose, calcium mesoxalate, miglitol or repaglinide. Non-traditional agents may include other saponins that in combination with an active compound exhibits a pharmacological response.

Combinations may be achieved by contacting cells with a single composition or pharmacological formulation that includes both agents (the active compound and anti-diabetic or anti-obesity agent), or by contacting the cell with two distinct compositions or formulations, at the same time. Alternatively, administration of one agent may precede or follow the treatment with a second agent by intervals ranging from minutes to weeks. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an active compound or the other anti-diabetic agent will be desired. Various combinations may be employed, where the active compound is “A” and the other agent or anti-diabetic is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated as well.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Panax ginseng and Panax quinquefolius Ginsenoside Analysis

Panax ginseng berry organic solvent extract was obtained from Jian Pharmaceutical Company, China. The constituents of the extract were analyzed using high performance liquid chromatography (HPLC). The high pressure gradient HPLC system was manufactured by Shimadzu Corp. (Kyoto, Japan). Chromatography was performed on a Phenomenex, Prodigy C18 5 μm 150×3.2 mm analytical column protected by guard column Phenomenex C8 30×3.2 mm. The dried powder (20 mg) was dissolved in 1,000 μl 90% MeOH, and 20 μl of solution was injected into the system. Separations were obtained by linear gradient elution, using eluents A (water) and B (acetonitrile) according to the following profile: 0-60 min, A 90-60%, B 10-40%, curve=1; 60-70 min, A 60%, B 40%. Flow rate was 0.6 ml/min at 22° C. The UV detector range was 0.01 AUFS and 202 nm wavelength.

Also, Panax quinquefolius berry and root extracts (obtained from Wisconsin) were extracted and analyzed using the same HPLC assay.

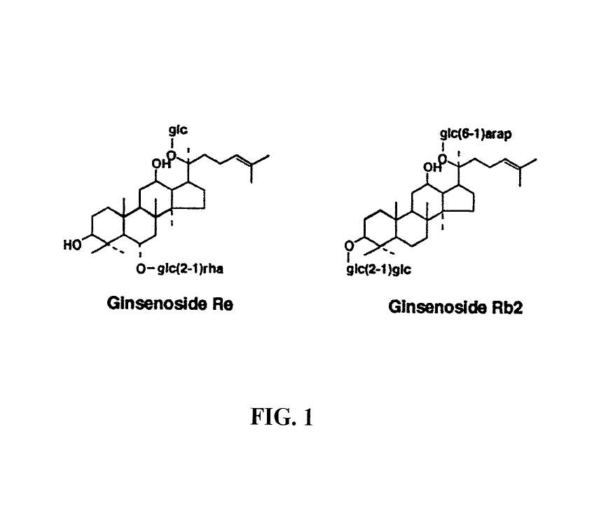

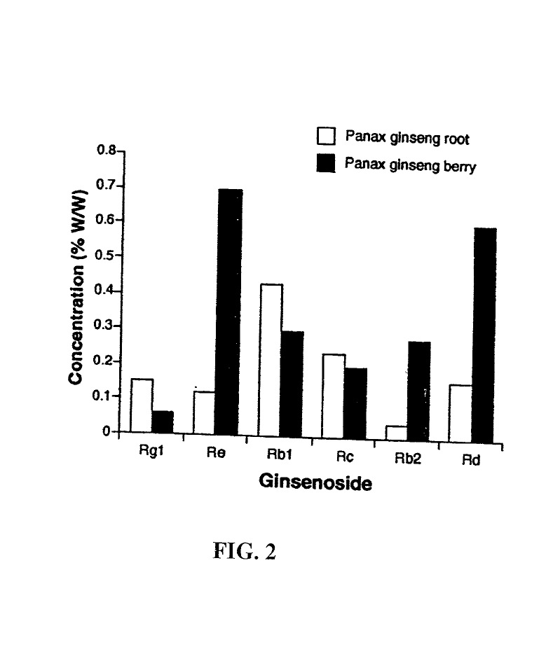

Panax ginseng root extract was also prepared using the same extraction procedure for the berry extract preparation. The root was obtained from Shanghai Pharmaceutical Company, China. FIG. 2 compares six major ginsenoside concentrations of Panax ginseng berry extract and Panax ginseng root extract using the same HPLC assay. FIG. 2 demonstrates that the concentrations of ginsenoside Re Rb2 and Rd in Panax ginseng berry are significantly higher than the root. Chemical structures of two ginsenosides are also shown in FIG. 1.

### Example 2

Preparation of Ginsenoside-Enriched and Ginsenoside-Free Panax ginseng Root Fractions

A procedure was developed at the University of Illinois Functional Food for Health Lab to successfully prepare ginsenoside-enriched and ginsenoside-free fractions from both methanol and water extracts prepared from Panax ginseng roots on request from a botanical supplements maker (Leiner Health Products, CA). The extracts were solubilized in water and chromatographed on a column of HP-20 (Diaion) and developed by a reverse gradient solvent system of H₂O (100%, fraction 1), H₂O-MeOH (8:2, fraction 2), H₂O-MeOH (1:1, fraction 3), MeOH (100%, fraction 4), and MeOH-CHCl₃ (1:1, fraction 5). Analysis by HPLC showed fractions to contain 0-60.1% total ginsenosides, and that the profiles of individual ginsenosides vary as follows: fraction 1 was devoid of ginsenosides; fraction 2 gave a total ginsenoside content of 6.0% with ginsenosides Rg1 (54.3%) and Re (35.5%) accounting for 90%; fraction 3 showed a total ginsenoside content of 60.1% with ginsenosides Rg1 (43.7%) and Re (24.4%) accounting for 68.1%; fraction 4 contained the second highest concentration of ginsenosides at 42.1%, with ginsenoside Rb1 accounting for 47% of the total; and fraction 5 showing trace quantities of ginsenosides. Thus, one of skill in the art will be able to utilize the above method for preparation of ginsenoside-enriched and ginsenoside-free fractions from ginseng berry extract.

### Example 3

Bioactivity-Guided Fractionation and Structure Identification/Elucidation

For the isolation of active isolates from active ginseng berry fractions, the materials are subjected to separation procedures employing gravity, vacuum, flash, and low-medium pressure column chromatography employing silica gel as the primary adsorbent will be used in the first order. Other adsorbents or media that may be employed include florisil, alumina, ion-exchange resins, exclusion gel (Sephadex), and C18-bonded silica gel. Preparative layer TLC or semi-preparative and/or preparative high performance liquid chromatography (Prep-HPLC) are used, especially in the isolation and purification of active compounds in the final stages of separation. Other systems of partition chromatography may also be employed as necessary.

The identification of known novel active isolates or the elucidation of their structure is established by measurement and interpretation of their physical (TLC Rf value, HPLC retention time, melting point, and mixture melting point) and spectroscopic (UV, IR, NMR and MS) data. In the identification of known active isolates, comparison of the melting point, optical rotation, UV, IR, 1H- and 13C-NMR, low resolution MS, TLC Rf and/or HPLC Rt values to those reported in the literature or to those obtained for reference standards are sufficient. For the elucidation of the structures of novel active compounds, the molecular formula is determined by exact mass measurement using a Q-TDF mass spectrometer) and/or elemental CHO(N) analysis. Tandem mass spectrometric fragmentation patterns coupled with appropriate NMR data allows the determination of functional groups and partial structures. Stereochemistry is determined by use of ORD and CD coupled with high-field 1H- and 13C-NMR spectroscopy, with appropriate two-dimensional (2D) and decoupling experiments (1H-1 H COSY, 1H-1H DQ-COSY and 1H-1H HOHAHA (TOCSY) for proton spin systems; 1H-13C HETCOR and HMQC for one-bond C-H couplings; HMBC, selective INEPT, 1H-13C COLOC and 1H-13C FLOCK for long range C-H correlations; NOESY, ROESY and NOE difference experiments for substituent spatial and stereochemical relationships). When necessary, chemical or enzymatic hydrolysis (e.g., ginsenosides and/or polysaccharides), and derivitization (e.g. acetylation, oxidation, reduction) experiments are conducted in conjunction with LC-MS-MS to assist in the determination of the structures. Conformational analysis may be performed using computer-aided molecular mechanics calculation (e.g. Monte Carlo searching method using MacroModel/Batchmin program (version 4.5) and MM2 forcefield (Sun et al., 1997, Qiu et al. 1998). In case of compounds with novel carbon skeletons or posing difficult stereochemistry problems, single crystal X-ray crystallographic analysis is carried out.

### Example 4

Panax ginseng Berry Extract Preparation and Administration

250 mg of Panax ginseng berry extract was dissolved in 50 ml MeOH as solution A. 1,500 mg polyvinylpyrrolidone or PVP-10 (Sigma Chemicals, St. Louis, Mo.) was dissolved in 50 ml MeOH as solution B. After mixing A and B, the mixture was evaporated under N₂ to yield 250 mg dried extract at 50° C. Before each experiment, the dried extract was dissolved in distilled deionized water and vortexed for 2 min at room temperature. The solution was injected intraperitoneally (IP) once a day at a dose of 50 or 150 mg/kg body weight. 150 mg/kg Panax ginseng berry extract contains approximately 20 mg/kg ginsenoside Re.

Also, ginsenoside Re was obtained from Shanghai Pharmaceutical Company, China. HPLC analysis was performed to confirm that ginsenoside Re had a purity of >99%. Ginsenoside Re, at a dose of 5, 10 or 20 mg/kg, was also dissolved in PVP-10 solution for daily IP administration.

Control treated animals were injected with an equimolar solution of PVP-10. No detectable irritation or restlessness was observed following each administration of the extract or vehicle.

### Example 5

Preparation of Panax quinquefolius Berry Extract

Panax quinquefolius (American ginseng) berry was collected in a private ginseng farm in Wausau, Wis. Fresh berry was first mixed 75% EtOH. The seeds were removed, and the pulp was collected and refrigerated. The pulp was filtered and refrigerated again. Then, the EtOH was evaporated. Distilled water was added to the solution, and filtered. The solution was further mixed with 1-BuOH, the water layer was then removed, and 1-BuOH was evaporated. Finally, the solution with extracts was lyophilized.

### Example 6

Preparation of a Polysaccharide Fraction from Panax quinquefolius Berry Extract

Fresh Panax quinquefolius (American ginseng) berry was obtained from a ginseng farm in Wausau, Wis. In brief, 500 g berry was mixed with 500 ml 75% EtOH. After removal of the seeds, 356 g pulp was collected. An additional 500 ml 75% EtOH was added, and the solution was heated and refluxed. This procedure was repeated three times. EtOH was then evaporated and the solution was filtered. The filtered solution contained both polysaccharides and ginsenosides.

The remaining solution was loaded onto a Dialon HP-20 gel column (Supelco, Va.), and the passed solution was collected. Afterwards, the column was washed with distilled water several times until the color of the solution disappeared. All collected solutions were mixed and filtered to obtain polysaccharides fraction (Sung et al., 2000). Finally, the extract was lyophilized. The extraction rate was approximately 2% from the fresh ginseng berry.

### Example 7

Animals

Male C57BL/6J ob/ob mice and their lean littermates (+/?) were obtained from Jackson Laboratory (Bar Harbor, Me.). In this obese, insulin-resistant mouse model, obesity is due to a mutation in the obese gene that encodes for leptin. Animals that are homozygous for the mutation exhibit morbid obesity and metabolic disorders that resembles type 2 diabetes in humans. The heterozygous mice are lean and normoglycemic. It is believed that the development of hyperglycemia in the ob/ob mouse is a consequence of epistatic interaction of the obesity mutation with diabetes-susceptibility genes. Adult animals at 10-18 weeks of age were used. Mice were housed in environmentally controlled conditions with a 12-h light/dark cycle and had free access to standard rodent pellet food, except when fasted before some experiments.

Another animal model, diabetic C57BL/KsJ db/db mice, also was utilized. C57BL/KsJ is an inbred strain distinct from the C57BL/6J strain, which serves as the recipient of the ob gene. In the C57BL/KsJ strain of mice, the diabetes db gene mutation occurred spontaneously (Shafrir, 1992). Male C57BL/KsJ db/db mice and their lean littermates (+/?) were obtained from the Jackson Laboratory (Bar Harbor, Me.). Adult animals at 10-15 weeks of age were used. Mice were housed in environmentally controlled conditions with a 12-h light/dark cycle and had free access to standard rodent pellet food, except when fasted before experiments.

### Example 8

Fasting Blood Glucose Level

Animals were treated with Panax ginseng berry extract, Panax quinquefolius berry extract, ginsenoside Re or a polysaccharides fraction from Panax quinquefolius and the fasting blood glucose levels were measured. Fasting blood glucose levels were measured after animals were fasted for 4 hr (starting from 9:00AM), on Day 0 (before treatment), Day 5 (during treatment), and Day 12 (last day of treatment). Blood glucose levels were determined in tail blood samples at 1:00PM using a Glucose Analyzer (Hemocue AB, Angelholm, Sweden).

Panax ginseng Berry Extract

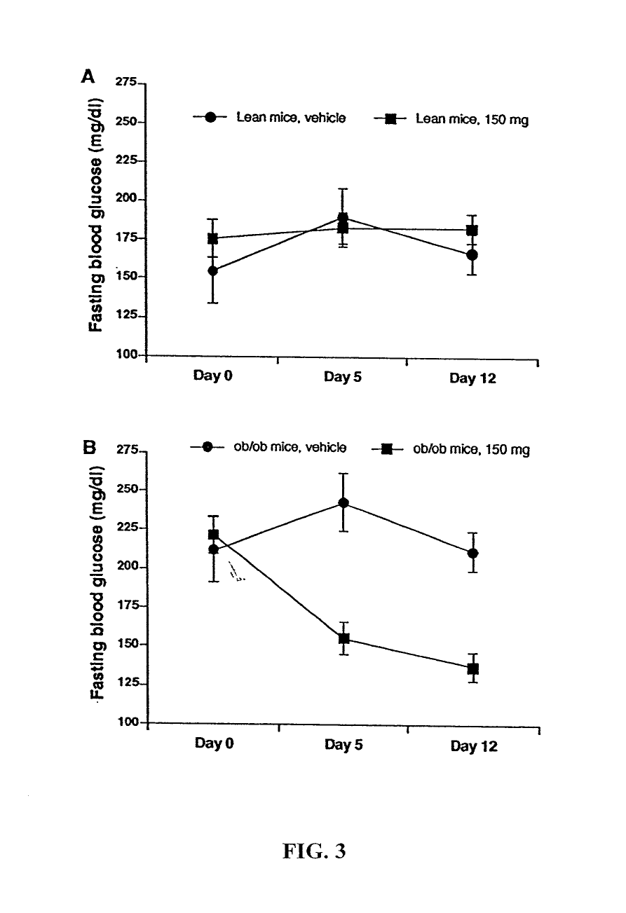

Blood glucose levels after 4 hr fasting in C57BL/6J ob/ob mice and their lean littermates were measured on Day 0, and on Day 5 and Day 12 after daily administration of Panax ginseng berry extract or vehicle. As shown in FIG. 3A and FIG. 3B, ob/ob mice had significantly higher fasting blood glucose levels compared to lean controls (222±16.2 vs. 176±12.1 mg/dl, P<0.01) on Day 0. On Day 5, blood glucose concentrations of ob/ob mice decreased significantly after treatment of Panax ginseng berry extract 150 mg/kg (156±9.0, P<0.01 with vehicle-treated mice 243±15.8 mg/dl). On Day 12, ob/ob mice treated with the extract were normoglycemic (137±6.7 mg/dl, P<0.01 compared with vehicle treated mice 211±19.6 mg/dl) and there was no significant difference in the levels between ob/ob mice and lean littermates (167±12.8 mg/dl). The blood glucose concentrations of lean mice did not change sizably in response to treatment with the extract (182±9.2 mg/dl vs. 167±12.8 mg/dl of vehicle-treated mice) in either of those days.

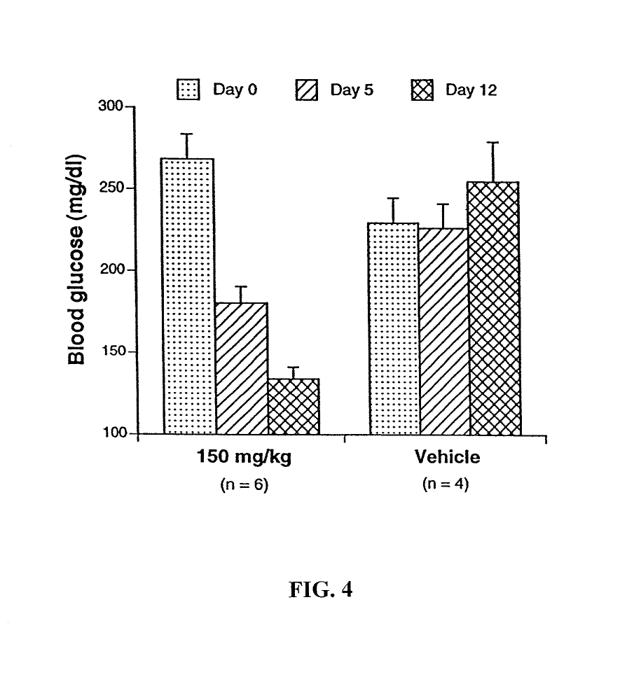

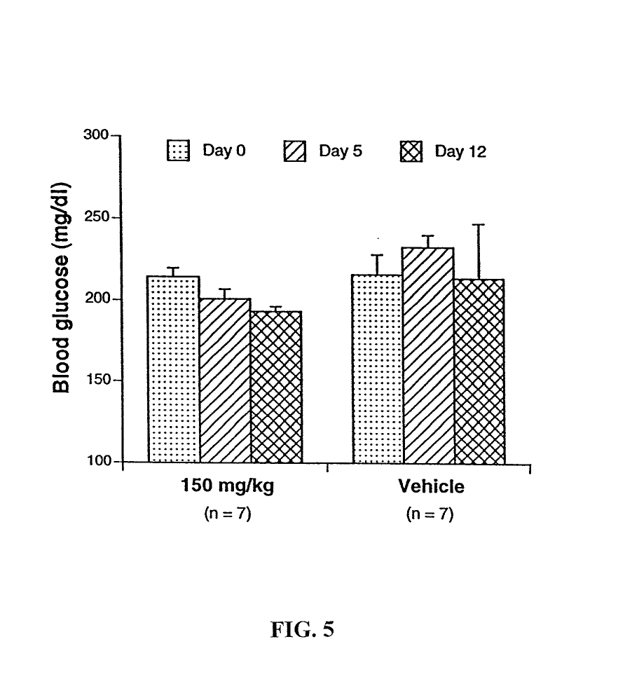

A second diabetic animal model was used to test the effects of Panax ginseng berry on fasting blood glucose levels. Four-hour fasting blood glucose levels were measured on Day 0, Day 5 and Day 12 after daily IP administration of Panax ginseng berry extract 150 mg/kg or vehicle, in db/db mice and their lean littermates. As shown in FIG. 4, db/db mice had higher blood glucose levels compared to the lean mice (FIG. 5) in the control condition. The extract markedly lowered incremental blood glucose level in db/db mice on Day 5 and Day 12. After daily administration of the extract for 5 days, blood glucose concentrations of db/db mice decreased significantly from 268±15.3 to 180.5±10.2 mg/dl (P<0.05 compared to the vehicle group of 226.0±15.3 mg/dl). After 12-day treatment, blood glucose level further reduced to 134.3±7.3 mg/dl and (P<0.01 compared to the vehicle group of 254.8±24.1 mg/dl) and returned to normoglycemic level. In lean littermates, there was a trend towards reduction in fasting blood glucose levels as seen in FIG. 5. However, the glucose concentration did not decrease significantly on Day 12 compared to the vehicle-treated mice (193±2.9 mg/dl vs. 213±7.4 mg/dl).

Panax quinquefolius Berry Extract

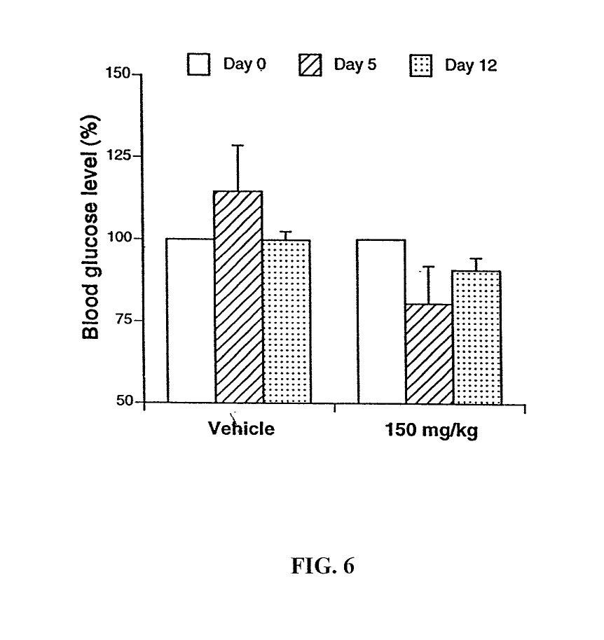

ob/ob mice received daily intraperitoneal (IP) injection of Panax quinquefolius ginseng berry extract 150 mg/kg or vehicle. On Day 0, ob/ob mice had high baseline fasting blood glucose levels (183.2±8.6 mg/dl in the extract-treated group and 212.0±14.9 mg/dl in vehicle-treated group). On Day 5 and Day 12, Panax quinquefolius ginseng berry extract 150 mg significantly decreased fasting blood glucose to 147.5±5.8 mg/dl and to 164.8±6.5 mg/dl, respectively (both P<0.05 compared to Day 0). In the vehicle-treated group, the fasting blood glucose levels did not change significantly (212.0±14.9 mg/dl on Day 0, 243.2±130.9 mg/dl on Day 5 and 211.6±20.8 mg/dl on Day 12). FIG. 6 shows percentage changes of fasting blood glucose levels after treatment, with Day 0 levels normalized to 100%.

Ginsenoside Re

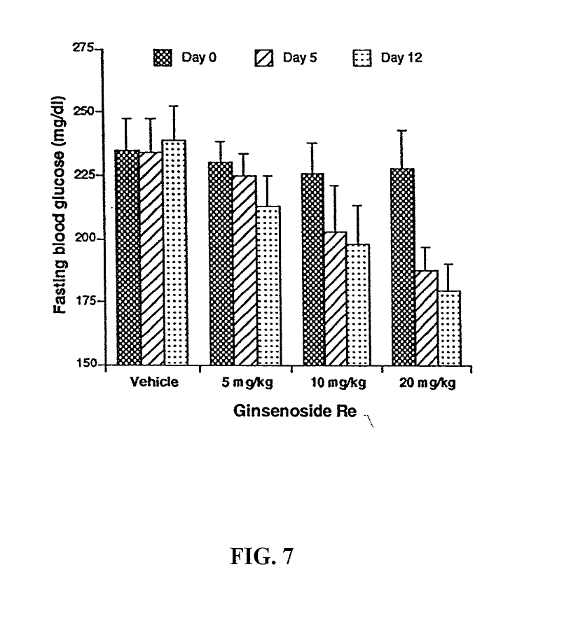

Blood glucose levels after 4 hr fasting were measured on Day 0, Day 5, and Day 12 after daily administration of ginsenoside Re. FIG. 7 shows dose-dependent effects of ginsenoside Re on fasting blood glucose in ob/ob mice. Fasting blood glucose concentrations decreased significantly after treatment with 20 mg/kg ginsenoside Re on Day 5 of 188±9.2 mg/dl and Day 12 of 180±10.8 mg/dl (both P<0.01 compared to vehicle-treated group on Day 5 of 234±13.7 mg/dl and Day 12 of 239±13.3 mg/dl). Fasting blood glucose concentrations did not change sizably in lean mice after treatment with ginsenoside Re.

Polysaccharides Fraction from Panax quinquefolius

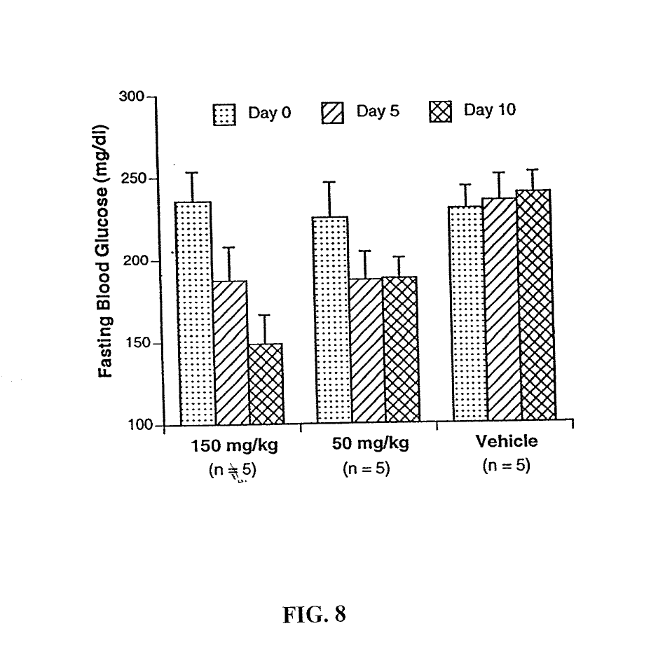

As shown in FIG. 8, ob/ob mice had remarkably high baseline fasting blood glucose levels. Polysaccharide fractions of Panax quinquefolius berry extract at doses of 50 mg and 150 mg significantly decrease fasting blood glucose levels. On Day 5, compared to the vehicle-treated mice (230.5±13.5 mg/dl), 150 mg/kg and 50 mg/kg polysaccharides-treated animals had significantly lower fasting blood glucose levels (187.4±20.5 mg/dl and 187.4±17.1 mg/dl, respectively; both P<0.05). On Day 10, compared to the vehicle group (240.1±12.3 mg/dl), 50 mg/kg polysaccharides-treated mice were 188.4±12.6 mg/dl (P<0.05), and 150 mg/kg polysaccharides-treated mice were normoglycemic (148.8±17.6 mg/dl, P<0.01). However, those ob/ob mice treated with vehicle did not show significant changes in fasting blood glucose levels.

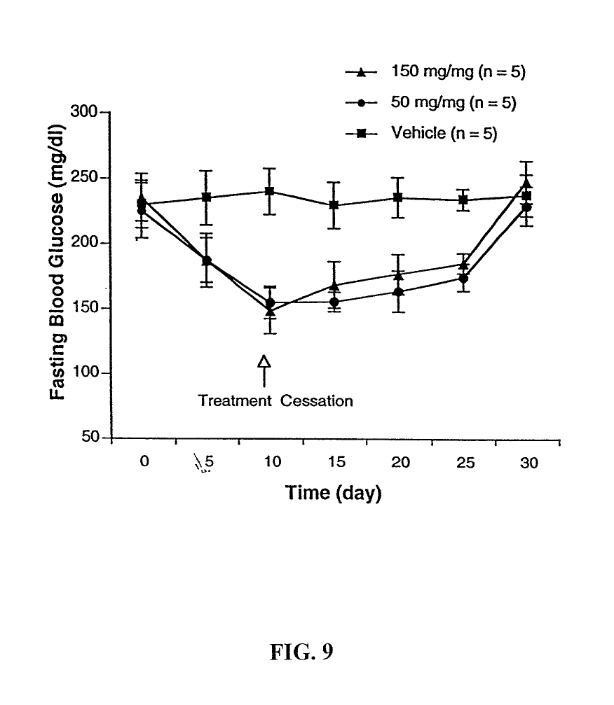

To observe whether there was an effect on fasting blood glucose concentration after cessation of polysaccharides treatment, the blood glucose was measured every five days until the levels returned to those prior to the treatment. FIG. 9 shows a prolonged effect in animals who received 150 and 50 mg/kg polysaccharides treatment. Fasting blood glucose levels were 168.6±17.7 and 155.6±7.4 on Day 15, 176.8±15.4, 163.8±15.7 on Day 20, and 185.6±7.9 and 174.8±10.4 on Day 25, respectively (all P<0.01 compared to the vehicle group).

Collectively, the above results clearly demonstrate that extracts of Panax ginseng berry, Panax quinquefolius berry, ginsenoside Re or polysaccharides from Panax quinquefolius significantly improved glucose homeostasis in diabetic mouse models; thus, these compounds are anti-hyperglycemic. Yet further, the data indicates that the effects of the compounds can last for an extended period of time. Thus, one skilled in the art will recognize that the above data demonstrate that ginseng berry extract, ginsenoside Re and polysaccharides can be used to treat diabetes.

### Example 9

Intraperitoneal Glucose Tolerance Test (IPGTT)

Animals were treated with Panax ginseng berry extract, Panax quinquefolius berry extract, ginsenoside Re, or a polysaccharide fraction from Panax quinquefolius . IPGTT was performed on Day 0 and Day 12. On the days of the test, animals were fasted for 4 hr (starting from 9:00AM) followed by an IP administration of glucose (2 g/kg). Blood glucose levels were determined in tail blood samples at 0 (prior to glucose administration), and 30, 60 and 120 min after glucose administration.

Panax ginseng Berry Extract

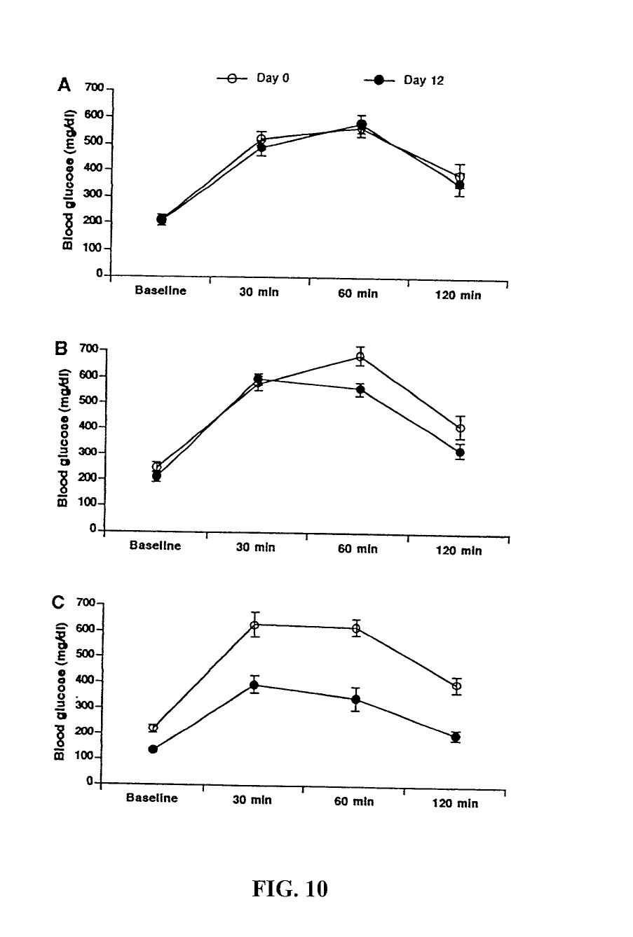

Glucose tolerance was evaluated by IPGGT, prior to and 12 days after treatment in ob/ob and lean mice with the extract or vehicle. As shown in FIG. 10, on Day 0, ob/ob mice demonstrated basal hyperglycemia, and this hyperglycemia was exacerbated by the IP glucose load, and failed to return to fasting level after 120 min, indicating glucose intolerance. After 12 days of treatment with Panax ginseng berry extract 50 mg/kg (FIG. 10B) and 150 mg/kg (FIG. 10C), the glucose tolerance of the ob/ob mice was dose-dependently improved. On Day 12, the blood glucose levels at 120 min following glucose administration approached to baseline (fasting) levels in 150 mg/kg extract treated ob/ob mice. The area under the curve (AUC) of blood glucose was decreased by approximately 46% compared to Day 0 in the 150 mg/kg extract-treated ob/ob mice group. This was a significant improvement in glucose exposure from 623 mg/ml·min of Day 0 to 334 mg/ml·min of Day 12 (P<0.01). In contrast, the glucose tolerance of lean control mice was unaffected by the both vehicle or 150 mg/kg extract.

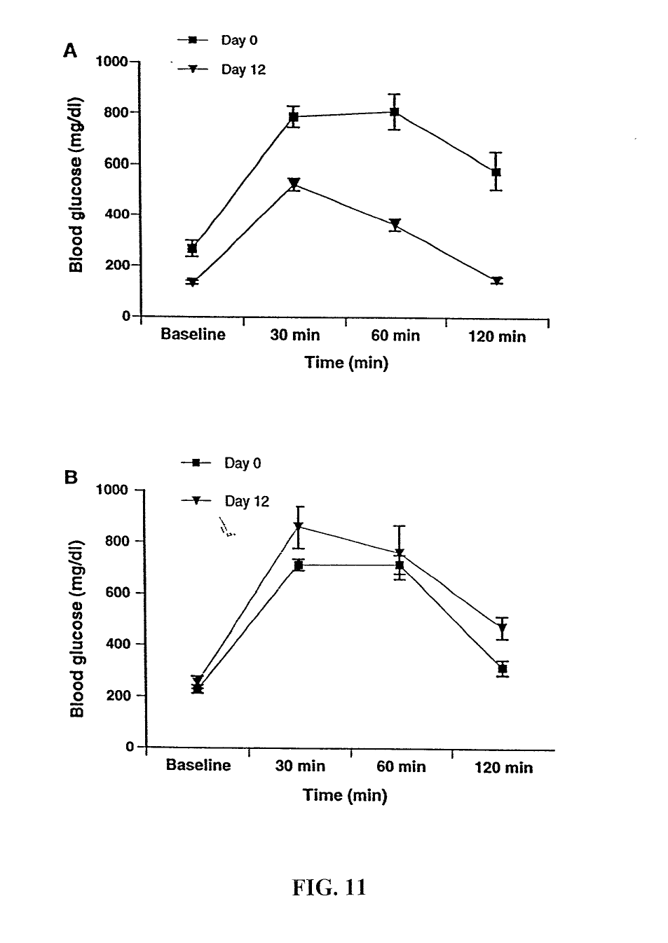

Yet further, another diabetic animal model was used to test glucose disposal after treatment with Panax ginseng berry. Glucose disposal was evaluated by IPGTT, prior to and 12 days post treatment with the extract or vehicle. As shown in FIG. 11, on Day 0, db/db mice demonstrated basal hyperglycemia, and this hyperglycemia was exacerbated by the IP glucose load, and did not return to baseline after 120 min indicating glucose intolerance and impaired disposal. After 12 days of treatment with Panax ginseng berry extract 150 mg/kg (FIG. 11A), there was a significantly higher rate of glucose disposal at 30, 60 and 120 min (P<0.01 compared to Day 0). FIG. 11B showed IPGTT data in db/db mice after treatment with vehicle, and no significant changes were seen. To evaluate the overall glucose exposure, the area under the concentration curve (AUC) was calculated. A significant improvement in glucose exposure was noted in the 150 mg/kg extract-treated db/db mice (Day 12), in which the AUC decreased 53.4% compared to Day 0 (from 814 g/L·min of Day 0 to 379 g/L·min of Day 12, P<0.01). In contrast, there was no significant change in the AUC in db/db mice who received vehicle (the AUC was 665 g/L·min of Day 0 and 781 g/L·min of Day 12).

Panax quinquefolius Berry Extract

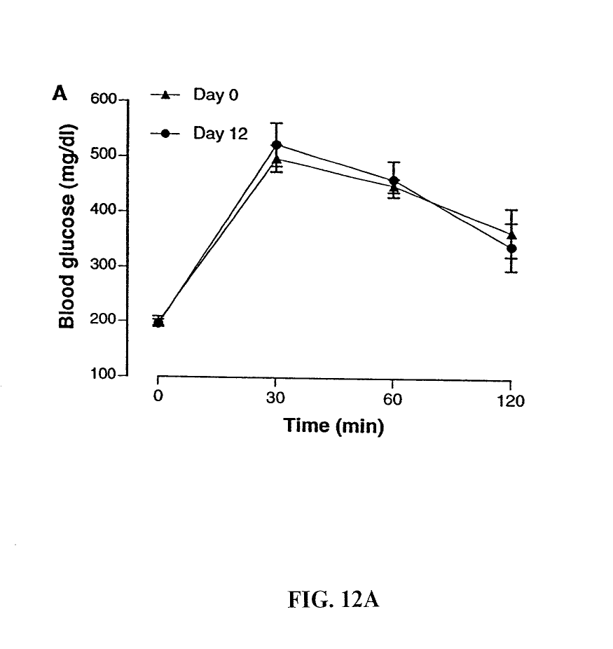

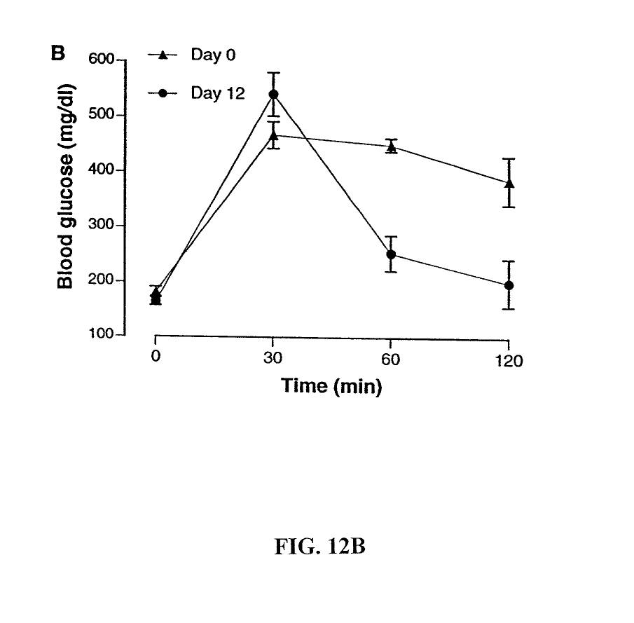

Glucose tolerance was evaluated by IPGTT, prior to and 12 days after Panax quinquefolius ginseng berry extract treatment. On Day 0, the hyperglycemia in ob/ob mice was exacerbated by the IP glucose load, and failed to return to baseline after 120 min, indicating glucose intolerance. Compared to treatment with vehicle (FIG. 12A), after 12 days of treatment with 150 mg/kg berry extract, the overall glucose exposure improved remarkably (FIG. 12B). The area under the curve (AUC) decreased 31.8% after treatment, from 646.6 mg/ml·min of Day 0 to 442.4 mg/ml·min of Day 12 (P<0.01). In vehicle group, however, no significant change was noted in the AUC between Day 0 and Day 12 (from 492.2 mg/ml·min of Day 0 to 497.0 mg/ml·min of Day 12).

Ginsenoside Re

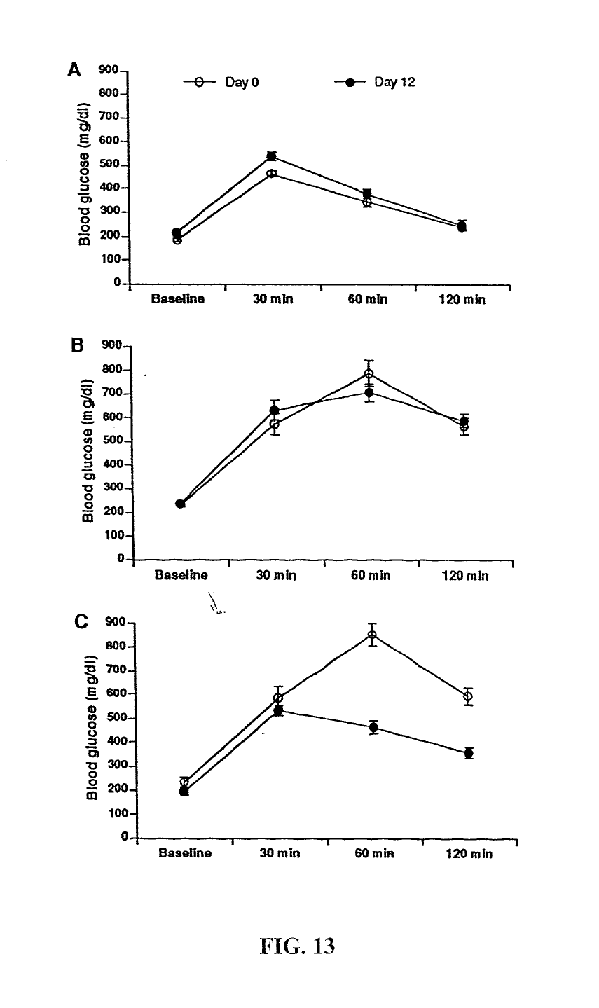

Ginsenoside Re was also administered at a dose of 150 mg/kg for 12 days to ob/ob mice. FIG. 13 shows glucose tolerance evaluated by IPGGT, prior to and 12 days after ginsenoside Re administration. FIG. 13A shows that with a 20 mg/kg ginsenoside Re treatment in lean mice the glucose tolerance was not statistically affected. In comparison, 20 mg/kg ginsenoside Re treatment in ob/ob mice (FIG. 13C) significantly decreased the blood glucose levels at 60 min and 120 min following glucose administration (both P<0.01) compared to vehicle-treated ob/ob mice (FIG. 13B).

Panax quinquefolius Polysaccharide Fraction

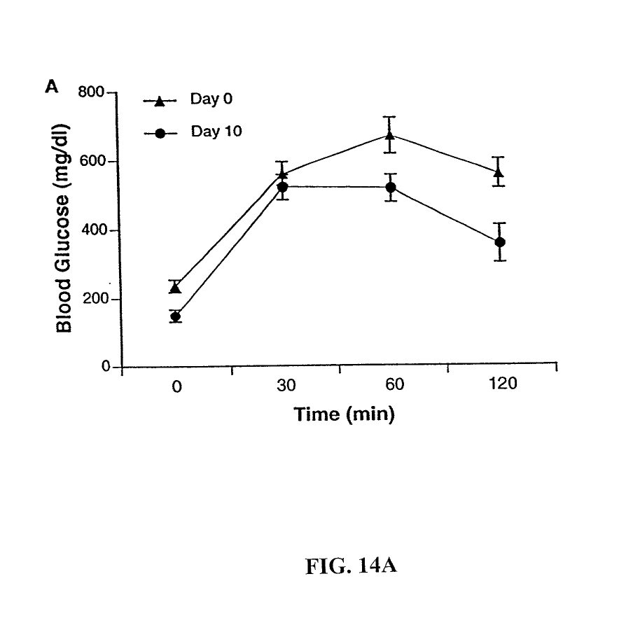

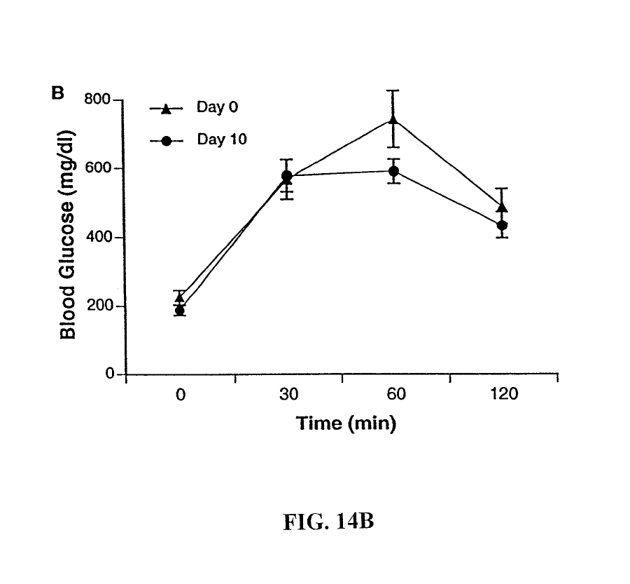

Glucose tolerance was evaluated by IPGTT, prior to and 10 days after treatment with the polysaccharides fraction. As shown in FIG. 14A (150 mg/kg group) and FIG. 4B (50 mg/kg group), on Day 0, ob/ob mice demonstrated basal hyperglycemia, and this hyperglycemia was exacerbated by the IP glucose load, and failed to return to baseline after 120 min indicating glucose intolerance. After 10-day treatment with polysaccharides (150 mg/kg and 50 mg/kg), the overall glucose tolerance improved remarkably. After 150 mg/kg and 50 mg/kg polysaccharides treatment, the area under the curve (AUC) decreased 28.2% (from 287.8 mg/ml·min of Day 0 to 206.7 mg/ml·min of Day 10, P<0.01) and 15.5% (from 249.6 mg/ml·min of Day 0 to 211.0 mg/ml·min of Day 10, P<0.05), respectively.

Thus, treatment with ginseng berry extracts, ginsenoside Re or a polysaccharide fractions of ginseng berry improves the ability of the ob/ob or db/db mice to handle a glucose load or metabolize glucose.

### Example 10

Hyperinsulinemic-Euglycemic Clamp

For the glucose clamp study, animals at Day 10 of treatment were catheterized in the right internal jugular vein under general anesthesia. The catheter was externalized through an incision in the skin flap at the vertex of the head. The catheterized animals were allowed to recover for 3-5 days under the continuous treatment of the extract or vehicle before the clamp studies. 120-min hyperinsulinemic-euglycemic clamps (6 mU/kg/min for lean mice, and 10 mU/kg/min for the ob/ob mice) were performed on 4 hr fasted mice by maintaining blood glucose concentrations at 6.6 mmol using a variable rate of 20% glucose infusion as previously described (Kim et al., 2000). During the clamps, mice were kept awake in mouse restrainers of proper size. Both glucose and insulin (porcine regular insulin, Eli Lilly, Indianapolis, Ind.) were administered into the same catheter implanted in the jugular vein through a T-connector. A two-channel micro-dialysis syringe pump (CMA/Microdialysis, Acton, Mass.) was used to control the rate of infusion. Blood samples were collected from the tail every 10 min during the clamps to measure the glucose levels and adjust the rates of glucose infusion. The average glucose infusion rate in the second half of the clamp was taken as the rate of whole body glucose disposal.

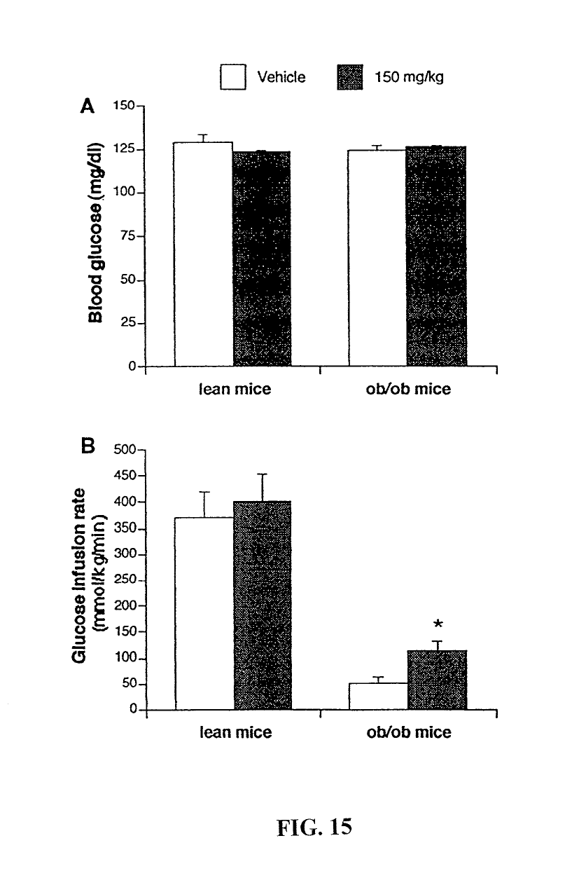

Body-wide insulin-stimulated glucose disposal rate with the hyperinsulinemic euglycemic clamp was measured. The rate of glucose disposal by the animals during the insulin stimulation was inferred from the amount of glucose infused per min to maintain blood glucose level at approximately 6.6 mmol. FIG. 15 shows blood glucose levels (FIG. 15A) and exogenous glucose infusion rates (FIG. 15B). Glucose infusion rate for untreated ob/ob mice was only approximately 18% of that in lean controls, indicating a severe peripheral insulin resistance. After a 12-day treatment with 150 mg/kg extract, the rate of insulin-stimulated glucose disposal in ob/ob mice was more than doubled relative to the vehicle-treated ob/ob mice (112±19.1 vs. 52±11.8 μmol/kg/min for the vehicle-treated group, P<0.01). Again, the extract did not affect the rate of glucose disposal in lean control mice (400±53.8 vs. 370±51.4, P<0.01).

Thus, the above data indicate that the ginseng berry extract improved peripheral insulin action. It is contemplated that improved peripheral insulin sensitivity will increase tissue glucose uptake and lower blood glucose levels.

### Example 11

Tissue-Specific Glucose Uptake

After the last blood sample in the hyperinsulinemic euglycemic clamp procedure, mice are sacrificed, and soleus muscle, extensor digitorum longus muscle, interscapular brown adipose tissue, and liver are excised and frozen in liquid nitrogen. The tissues are digested by NaOH and deproteinated as previously described (Wang et al., 1999) or by the Somogyi procedure. The supernatant from the deproteinated samples is mixed with scintillation cocktail, and ¹⁴C radioactivity will be quantitated (Hom et al., 1984; Ferre et al., 1986). After the plasma samples are deproteinated using the Somogyi procedure, an aliquot of the supernatant is evaporated overnight, and then mixed with scintillation cocktail for determination of [¹⁴C]2-DG with a liquid scintillation counter. The rate of glucose uptake in the muscle, adipose tissue and liver is calculated according to Kraegen's method (Kraegen et al., 1982).

### Example 12

Monitoring of Food Consumption

Animals were individually housed in a specially designed Metabolic Cage (Nalge Nunc International, Rochester, N.Y.), which has a food chamber that only permits the insertion of the head. The cage also has a deck to collect spilled food pellets without contamination. Food intake was determined by measuring the difference between the pre-weighed standard chow and the weight of chow and spill every 24 hours.

During 12-day observation in ob/ob mice, the mean daily food intake of vehicle group and 150 mg/kg extract-treated group were 4.7±0.1 g and 3.0±0.1 g, respectively. There was a significant difference in the mean daily food intake between the vehicle group and 150 mg/kg extract-treated group (P<0.01).

### Example 13

Body Weight

The average body weight of adult ob/ob or db/db mice is almost twice that of their lean littermates. Animals were treated with Panax ginseng berry extract, Panax quinquefolius berry extract, ginsenoside Re, or a polysaccharide fraction from Panax quinquefolius and the body weights were determined.

Panax ginseng Berry Extract

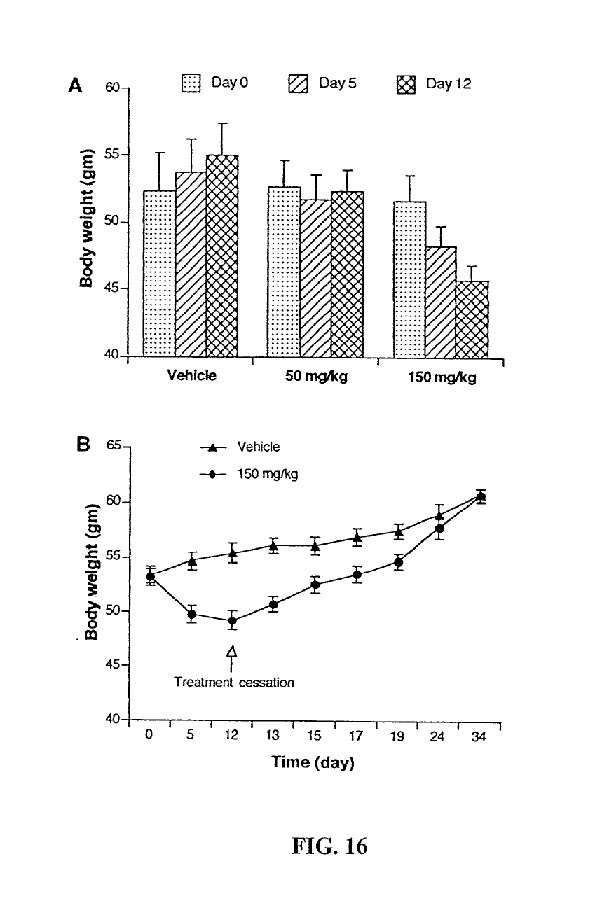

FIG. 16A shows the effects of Panax ginseng berry extract on body weight changes in ob/ob mice. The body weight of animals in the vehicle-treated group showed a tendency to increase from Day 0 to Day 12. During a 12-day treatment with extract at 50 mg/kg, body weight increase ceased. However, after a 12-day treatment with extract at 150 mg/kg, body weight reduced significantly from 51.7±1.9 g on Day 0, 48.3±1.5 g on Day 5, to 45.7±1.2 on Day 12 (P<0.05 and P<0.01 compared to Day 5 and Day 12 vehicle-treated ob/ob mice, respectively). Following the cessation of treatment, ob/ob mice gradually regained weight, and their body weight approached that of vehicle treated ob/ob mice after 22 days (FIG. 16B).

The body weight of lean mice in vehicle-treated group also showed a tendency to increase from 27.1±1.2 g on Day 0, 27.8±1.9 g on Day 5, to 28.9±1.0 g on Day 12. However, during a 12-day treatment with extract at 150 mg/kg, body weight increase in lean mice ceased, i.e., 26.5±1.5 g on Day 0, 26.9±1.4 g on Day 5, and 26.5±1.0 g on Day 12.

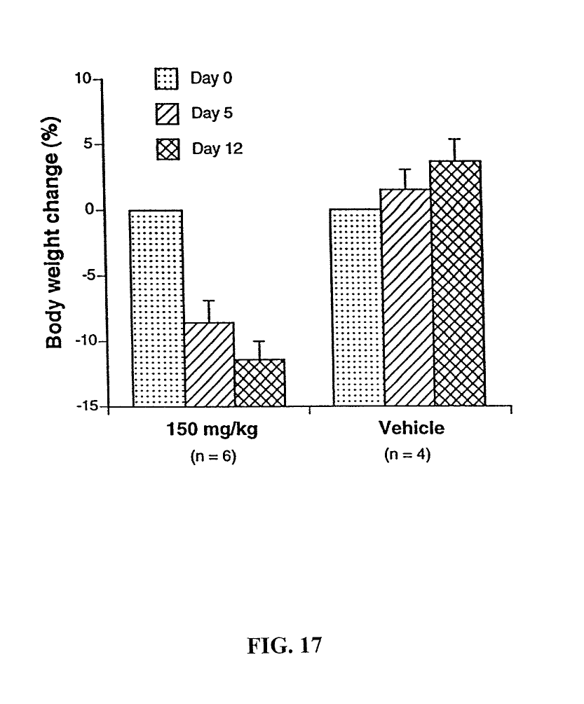

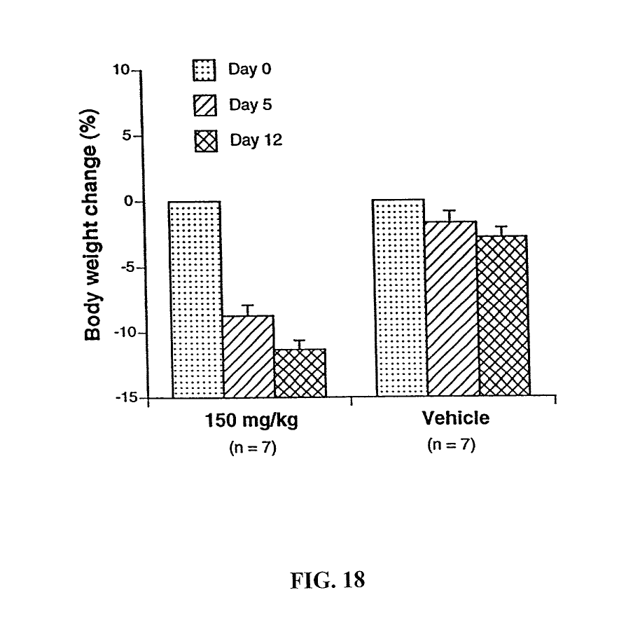

Another diabetic model (db/db) also was examined for anti-obesity effects of Panax ginseng berry extract. After the extract 150 mg/kg treatment, there were significant body weight reductions in both db/db mice (FIG. 17) and their lean littermates (FIG. 18). After 5 and 12 days of extract treatment, body weight in db/db mice reduced from 51.0±1.9 g on Day 0, to 46.6±1.7 g on Day5, and to 45.2±1.4 g on Day 12(P<0.05 and P<0.01 compared to Day 0, respectively). Similar decreases in body weight were observed in lean mice treated with the extract (from 30.9±0.8 g on Day 0, to 28.2±0.7 g on Day 5, to 27.4±0.7 g on Day 12; both P<0.01 compared to Day 0, respectively).

Panax quinquefolius Berry Extract

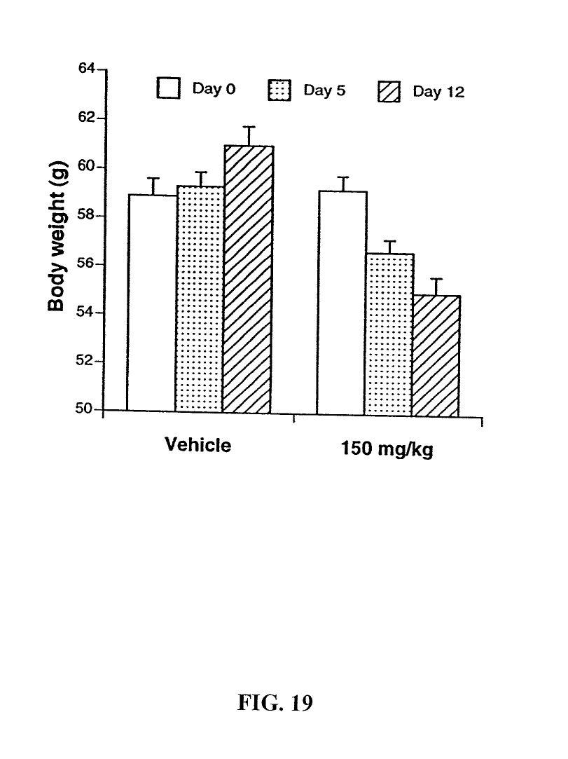

As shown in FIG. 19, the body weight of vehicle-treated ob/ob mice had a tendency to increase from Day 0 to Day 12 (58.9±0.7 g on Day 0, to 59.3±0.6 g on Day 5, and to 61.0±0.8 g on Day 12). Panax quinquefolius ginseng berry extract 150 mg/kg significantly decreased body weight in these obese mice. After 5 and 12 days treatment with extract, body weight reduced from 59.2±0.6 g on Day 0, to 56.7±0.5 g on Day 5, and to 55.0±0.7 g on Day 12 (both P<0.01 compared to Day 0).

Ginsenoside Re

In contrast to both anti-diabetic and anti-obese effects of Panax ginseng berry, ginsenoside Re did not result in a reduction in body weight. After 12-day treatment with ginsenoside Re 20 mg/kg, body weight did not change significantly in ob/ob mice. Body weight in ginsenoside Re 20 mg/kg group was 53.1±1.4 g on Day 0, 52.9±1.5 g on Day 5, and 54.7±1.7 on Day 12.

Panax quinquefolius Polysaccharide Fraction

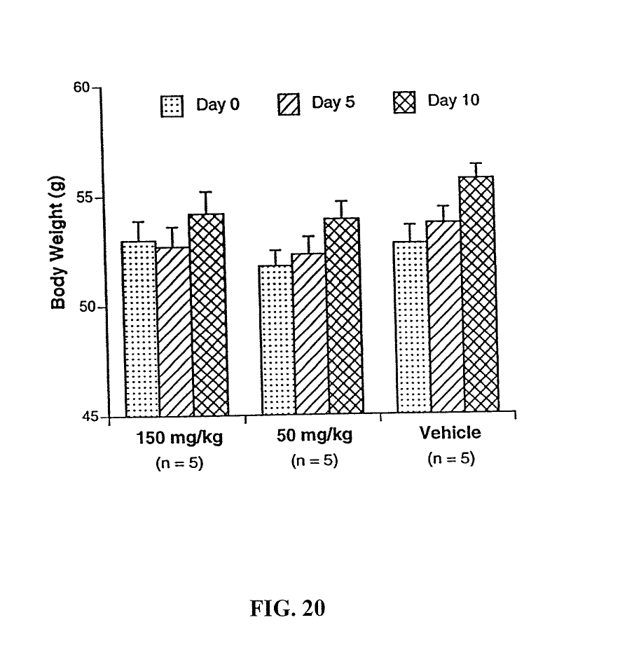

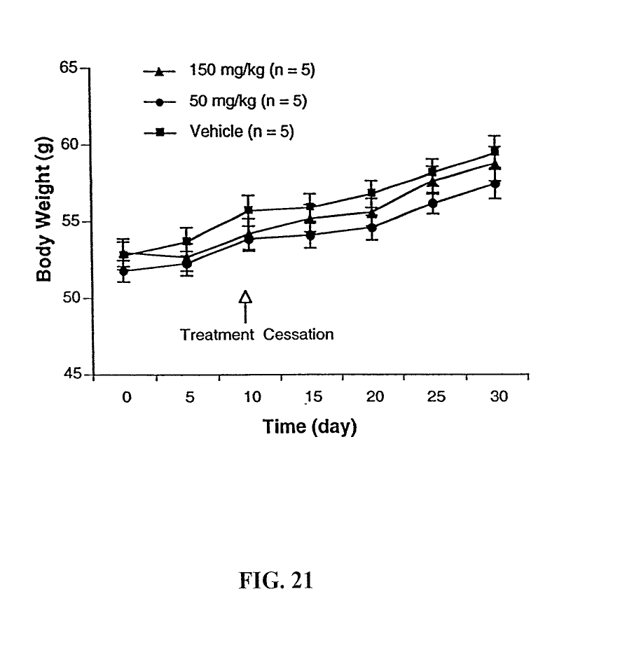

As shown in FIG. 20, body weight of ob/ob mice in the vehicle-treated group had a tendency to increase from Day 0 to Day 10. This tendency of body weight increase was not affected by 150 mg/kg or 50 mg/kg polysaccharides administration. However, in 30 days observation, body weight changes were not affected by polysaccharides treatment (FIG. 21).

Thus, similar to ginsenoside Re, polysaccharide fractions of the berry extract only exhibited significant anti-hyperglycemic effects. Thus, this data indicates that the anti-hyperglycemic effects of ginsenoside Re and polysaccharide fractions of berry extracts are independent of body weight changes. Yet further, this data suggests that other constituents in the berry extract have distinct pharmacological mechanisms that affect energy metabolism.

Also, the above data indicate that the ginseng berry extracts clearly are anti-hyperglycemic and anti-obesity agents.

### Example 14

Body Temperature and Energy Expenditure

Mice body temperature was measured with a thermocouple probe (Physitemp, Clifton, N.J.). On Day 0, Day 5 and Day 12 at 3:00PM, the thermocouple probe was inserted approximately 1 cm into the rectum to obtain body temperature.

Oxygen consumption measurements were made in an Oxymax chamber with an air flow rate of 0.181/min for 2 hr at 25° C. Airflow was controlled and measured using a mass flow meter (Flow control [R-1], Applied Electrochemical Inc., Pittsburgh, Pa.). Gas composition of incoming outdoor air and exhaust gas were measured using an infrared gas analyzer for CO₂ (Infrared Analyzer 864, Beckman Instruments, Fullerton, Calif.), and an electrochemical O₂ detector (Ametek S-3A, Applied Electrochemical Inc., Pittsburgh, Pa.). Gas analyzers were calibrated daily using cylinders of primary gas standard mixtures with known concentrations of CO₂, O₂ and N₂. Each animal was placed in a respiration chamber and was allowed to equilibrate for 1 hr. Oxygen consumption and CO₂ production were monitored every 5 min during the 2nd hr with the use of a computer-controlled open circuit calorimetry system. Values for energy expenditure (Elia and Livesey, 1992) were calculated every 5 min. Instruments were interfaced with a computer for calculations.

As expected, ob/ob mice were significantly hypothermic (35.6±0.2° C.) compared to their lean littermates (36.9±0.2° C., P<0.01). After 12-day treatment with 150 mg/kg extract, body temperature in ob/ob mice significantly increased from 35.6±0.1° C. (Day 0) to 36.6±0.1° C. (Day 12, P<0.01).

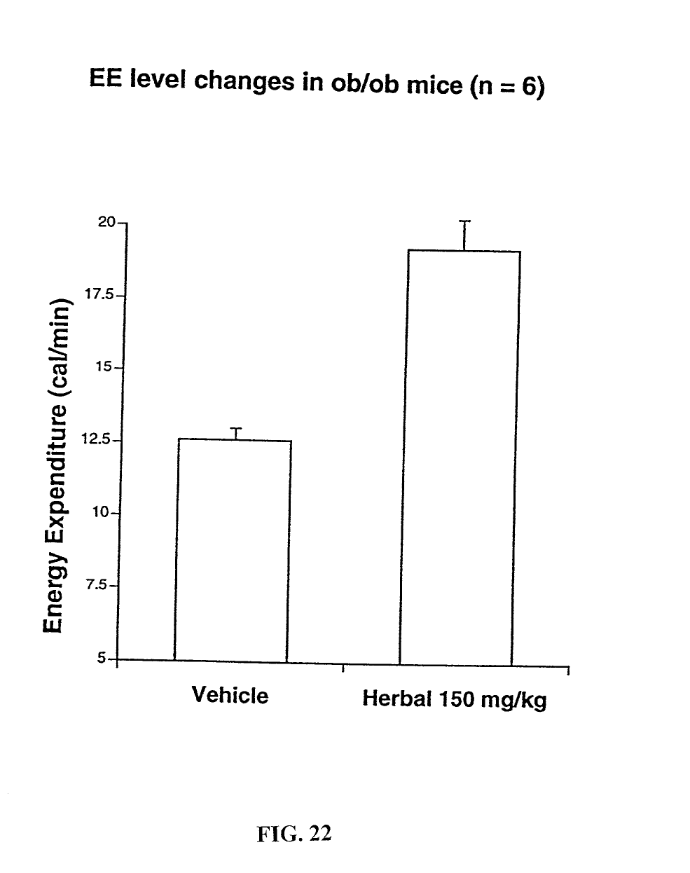

Energy expenditure values were obtained in ob/ob mice treated with vehicle or Panax ginseng extract 150 mg/kg (FIG. 22). After the 12-day treatment, there was a significant increase in energy expenditure of the extract-treated group compared to the vehicle-treated group (19.3±1.0 cal/min vs. 12.6±0.4 cal/min, P<0.01).

### Example 15

Plasma Cholesterol Changes

Panax ginseng berry extract also significantly reduced plasma cholesterol levels in ob/ob mice (FIG. 23). Plasma cholesterol concentration of 150 mg/kg extract-treated ob/ob mice was significantly lower (117±18.3 mg/dl) compared to the vehicle-treated animals (169±12.4 mg/dl, P<0.05).

### Example 16

Metabolic Parameters

Additional metabolic parameters are measured using techniques well known in the art. For example, analysis of biochemical parameters, such as, insulin, triglyceride, cholesterol, free fatty acids, and glycerol are performed spectrophotometrically on a clinical chemistry analyzer. Insulin is measured by radioimmunoassay (Cambridge Diagnostics, Billerica, Mass.).

### Example 17

Ginseng Berry Fractions and Blood Glucose

For studies with fractions, oral administration (3, 10, 30 and 100 mg/kg) is used. Troglitazone (30 mg/kg in rats; Luo et al., 1998) is used as a positive control. Troglitazone is a currently used oral agent that improves peripheral insulin sensitivity.

The HPLC fractionation, purification, and quantification studies are performed as previously described in the present invention.

The effect of ginseng berry fractions on fasting blood glucose is examined in adult ob/ob mice, and lean C57BL/6J mice that are weight-matched and divided into 6 groups. Each group is treated with a daily oral ginseng berry fraction (50, 150 mg/kg) or vehicle for 12 days. After a 4 h fast, on day 0 (before treatment), 5, and day 12, 10 μl blood is collected from the tail vein for measurement of fasting glucose level. Intraperitoneal glucose tolerance test (IPGTT) is done on day 0 (before treatment) and day 12 (last day of treatment). On the day of the test, animals are fasted for 4 hr (starting from 9:00AM) followed by an intraperitoneal injection of glucose (2 g/kg). Blood glucose levels are determined in blood samples from the tail vein at 0 (prior to), and 30, 60 and 120 min after glucose administration with a Glucose Analyzer (Hemocue AB, Angelholm, Sweden).

It is contemplated that some of the ginseng berry HPLC fractions will give variable effects with the fasting blood glucose test and glucose tolerance. Previous studies with type 1 diabetic mice have shown that glycan fractions of Panax ginseng root have significant anti-hyperglycemic activity. In type 2 diabetes models, hyperglycemia is mainly due to insulin resistance, and may not be related to defects in insulin secretion (as seen in type 1 model).

### Example 18

Diabetic Patients Treated with Extract or Active Compound

For clinical studies, the whole berry extract, compositions comprising at least one ginsenoside or compositions comprising more than one ginsenoside, and/or an organic extracts comprising at least one ginsenoside are administered to a subject suffering from hyperglycemia and/or type 2 diabetes. Ginsenosides are highly lipid soluble, thus an organic solvent can be used to solublize the ginsenosides.

Administration of the extract or active compound comprises a variety of routes depending upon the form of the extract or compound. For example, whole berry extract is administered orally. Administration of the compositions comprising a single ginsenoside or compositions comprising multiple ginsenosides is administered subcutaneously. Subcutaneous administration includes, but is not limited to, a single injection, multiple injections or continuous infusion. And administration of the organic extract or the compositions comprising a one or more than one ginsenoside is administered transdermally via a patch. In addition to administering the extract or the active compound, a placebo compound is administered to subjects via similar routes.

For the experiments, the subjects (diabetic) are treated with at least a daily dose of the compound or placebo for a period of time, e.g., 5 days, 12 days, 14 days, 21 days or longer.

The endpoint of the treatment period comprises measurement of blood glucose levels via a variety of techniques that are well known and used by clinical laboratories. Such measurement comprise hemoglobin A_{Ic} (HbA_{Ic}), fasting blood glucose and/or glucose tolerance. For all of these measurements, blood is drawn from the subjects that were treated with the compound and subjects that were treated with the placebo.

One of skill in the art is cognizant that the best characterized variant of hemoglobin is A_{Ic}, which constitutes about 3.5% of the hemoglobin in a normal subject, but may be increased two to three fold in individuals suffering from diabetes. HbA_{Ic} is formed by nonenzymatic glycosylation of hemoglobin A. Its level is directly proportional to the time-integrated mean blood glucose concentration over the preceding two to three months. For this reason, this measurement is widely used as a measure of glucose control in diabetic patients and is a tool for diagnosis of diabetic states. Normal and variant hemoglobins can be detected and quantified by standard clinical laboratory techniques. HbA_{Ic} is expressed as the percentage of total hemoglobin. For example, in a normal subject, the percentage of HbA_{Ic} is about 3.3% and for a diabetic patient it is about 8% or higher. The advantage of using HbA_{Ic} is that it is not influenced by acute changes in blood glucose or by the interval since the last meal. Thus, it is contemplated that the HbA_{Ic} percentage will decrease in diabetic subjects that are treated with the extract or active compound compared to diabetic subjects treated with a placebo. It is further envisioned that with continued usage of the ginseng berry extract or active compound that the levels of HbA_{Ic} in a diabetic subject will be similar to a normal subject, thus illustrating that the ginseng berry extract or active compound can function to maintain glucose homeostasis in a diabetic subject.

Fasting blood glucose and oral glucose tolerance tests are preformed similar to the procedures that are well known in the art. Briefly, after a 4 h fast on day 0 (before treatment), 5, and day 12 or any other endpoint, blood is collected for measurement of fasting glucose level. Oral glucose tolerance test is done on day 0 (before treatment) and day 12 (or last day of treatment). On the day of the test, subjects are fasted for 4 hr followed by an oral consumption of glucose (usually 30-50 g). Blood glucose levels are determined in blood samples from at 0 (prior to), and 30, 60 and 120 min after glucose administration with a Glucose Analyzer (Hemocue AB, Angelholm, Sweden). It is envisioned that the subjects treated with the extract or the active compound will have decreased fasting blood glucose levels compared to that of diabetic subjects treated with a placebo. Also, subjects treated with the extract or the active compound are envisioned to have improved glucose tolerance.

### Example 19

Patients Treated with Anti-Obesity Agents

For clinical studies, the whole berry extract, compositions comprising at least one ginsenoside or compositions comprising more than one ginsenoside, and/or an organic extracts comprising at least one ginsenoside are administered to a subject suffering from obesity. Ginsenosides are highly lipid soluble, thus an organic solvent can be used to solublize the ginsenosides.

Administration of the extract or active compound comprises a variety of routes depending upon the form of the extract or compound. Whole berry extract is administered orally, for example, as a solution or tablet. Administration of the compositions comprising a single ginsenoside or compositions comprising multiple ginsenosides are administered subcutaneously. Subcutaneous administration includes, but is not limited to, a single injection, multiple injections or continuous infusion. And administration of the organic extract or the compositions comprising a one or more than one ginsenoside is administered transdermally via a patch.

Obese subjects are treated with at least a daily dose of the compound for a period of time, e.g., 5 days, 12 days, 14 days, 21 days or longer.

Prior to treatment, the obese subjects are weighed to determine the starting body weight. In addition to starting body weight, metabolic parameters are also measured. Metabolic parameters include, body temperature, energy expeniture, cholesterol, free fatty acids, glycerol, insulin, and triglyceride. The mesurement of these parameters are well known and used in the clinical environment.

During the treatment, body weight and metabolic parameters are measured to determine the effects of the treatment of ginseng berry extract or compositions on obese subjects. It is anticipated that ginseng berry extract or compositions will decrease body weight in obese subjects. A decrease in body weight may be related to increases in body temperature or energy expeniture and/or decreases in food consumption. Thus, ginseng berry extract or compositions may be used as an anti-obesity agent for these subjects.

### Example 20

Patients Treated Treated with Extract or Active Compound Decrease Cholesterol Levels

For clinical studies, the whole berry extract, compositions comprising at least one ginsenoside or compositions comprising more than one ginsenoside, and/or an organic extracts comprising at least one ginsenoside are administered to a subject suffering from high cholesterol. Ginsenosides are highly lipid soluble, thus an organic solvent can be used to solublize the ginsenosides.

Administration of the extract or active compound comprises a variety of routes depending upon the form of the extract or compound. For example, whole berry extract is administered orally. Administration of the compositions comprising a single ginsenoside or compositions comprising multiple ginsenosides is administered subcutaneously. Subcutaneous administration includes, but is not limited to, a single injection, multiple injections or continuous infusion. And administration of the organic extract or the compositions comprising a one or more than one ginsenoside is administered transdermally via a patch. In addition to administering the extract or the active compound, a placebo compound is administered to subjects via similar routes.

For the experiments, the subjects are treated with at least a daily dose of the compound or placebo for a period of time, e.g., 5 days, 12 days, 14 days, 21 days or longer.

Prior to the treatment, blood is drawn from the subjects to determine the baseline cholestrol level. Next, the subjects are treated for a period of time with the compound or extract and their cholesterol levels are measured.

It is envisioned that treatment with berry extracts or compositions may decrease plasma cholesterol levels. Thus, the compositons of the present invention may be an anti-cholesterol agent.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

Abraira et al., (1995) Diabetes Care 18:1113-1123.

Attele et al., (1999) Biochem. Pharmacol. 58:1685-1693.

Bailey (1999) Biochem Pharmacol 58:1511-1520.

Banthorpe, In: Natural Products. Longman Scientific and Technical, Essex, 1994.

Bastiaanse et al., (1997) Cardiac Res 33 :272-283.

Boar, Terpenoids and Steroids. Bartholomew Press, Dorking, England, 1983.

Bonadonna et al., (1993) J Clin Invest 92:486-494.

Brownlee et al., (1984) Ann Intern Med 101:527-537

Chung et al., (1998) Steroids 63:421-424.

Davydov et al., (1990). Patologicheskaia Fiziologiia i Eksperimentalnaia Terapiia 5:49-52.

Defronzo (1985) et al., J Clin Invest 76:149-155.

Elia and Livesey (1992). World Rev Nutr Diet 70:68-131.

Eriksson et al., (1989). N Eng J Med 321:337-343.

Ferrannini (1998). Endocrine Rev 19:477-490.

Ferrannini et al., (1989). Diabetes Metab Rev 5:711-725.

Ferre et al., (1986). Biochem J 233:249-252.

Firth et al., (1986). J Clin Invest 77:1525-1532.

Garvey et al., (1991) J Clin Invest 87:1072- 1081.

Gaster and Hirsch (1998) Arch Intern Med 158:134-140.

Gillis (1997) Biochem Pharmacol 54:1-8.

Ginsberg et al., (1975) J Clin Invest 55:454-461.

Guma et al., (1995) Am J Physiol 268:E613 -622.

Hasegawa et al., (1995) Planta Med 61:409-413.

Heftmann and Mosettig, Biochemistry of Steroids, Reinhold Publishing Corp., London, 1960.

Hom et al., (1984) Diabetes 33:141-152.

Horikawa et al., (2000) Nature Genetics 26:163-175.

Huang et al., The Pharmacology of Chinese Herbs, CRC Press, Boca Raton, Fla., 1999.

Ihara (1999) Diabetes 48:927-932.

Islam et al., (1997) Drug Saf 17:149-165

Karikur et al., (1991) Chem Parm Bull (Tokyo) 39:2357-2361.

Kashiwagi et al., (1983) J Clin Invest 72:1246-1254.

Kelley et al., (1996) J Clin Endocrinol Metab 77:1287-1993.

Kim et al., (1998) J Basic Clin Physiol Pharmacol 9:325-345.

Kim et al., (2000) J Biol Chem 275:8456-8460.

Kimura (1980). Hypoglycemic component in ginseng radix and its insulin release.

Proceedings of the 3rd International Ginseng Symposium. Korean Ginseng Research Institute, Seoul, Korea.

Kimura amd Suzuki (1981). J Pharm Dyn 4:907-915.

Kimura et al., (1981a). J Pharm Dyn 4:410-417.

Kimura et al., (1981b). J Pharm Dyn 4:402-409.

Kimura et al., (1999). Phytotherapy Research 13:484-488.

Klein and Klein (1998). Diabetes Care 21 (Suppl. 3):C39-C43.

Klein et al., (1994). Arch Intern Med 154:2169-2178.

Kraegen et al., (1982). Am J Physiol 245:E1-E7.

Kruszynska and Olefsky (1996). J Invest Med 44:413-428.

Kudo et al., (1998) Eur J Pharmacol 341:139-144.

Lee, Facts about Ginseng, the Elixir of Life, Hollyn International Corp., Elizabeth, N.J., 1992.

Lillioja et al., (1993). N Eng J Med 329:1988-1992.

Liu et al., (1993). Diabetologia 36:428-432.

Luo et al., (1998). Diabetic Med 15:367-374.

Mitrakou et al., (1990). Diabetes 39:1381- 1390.

Molokovskii and Barnaulov (1986). Rastitel'Nye Resursy 22:387-393.

Molokovskii et al., (1989). Problemy endokrinologii 35:82-87.

Odani et al., (1983) Chem Pharm Bull (Tolkyo) 31:1059-1066.

Ohkubo et al., (1995). Diabetes Res Clin Pract 28:103-117.

Okuda and Yoshida (1980). Studies on the effects of ginseng components on diabetes mellitus. Proceedings of the 3rd International Ginseng Symposium, Korean Ginseng Research Institute, Seoul, Korea.

Ota et al., (1987) Cancer Res 47:3863-3867.

Ourisson et al., Tetracyclic Triterpenes. Holden-Day, Inc. San Francisco, 1964.

Qiu, S-X (1998). Carbohydrate Res. 311:85-88.

Reaven (1983). Am J Med 74:3-17.

Rubin et al., (1994). J Clin Endocrinol Metab 78:809A-809F.

Sakurai and Tsuchiya (1988). FEBS Lett 236:406-410.

Seely et al., In: Moller D, ed. Insulin resistance and its clinical disorders. Chichester: John Wiley & Sons, Ltd, 1993:187-252.

Shafrir (1992) Diabetes Metab Rev. 8:179-208.

Shen et al., (1970). J Clin Invest 49:2151-2160.

Shepherd et al., In: Moller D E, ed. Insulin resistance. Chinchester:John Wiley and Sons ltd. 1993; 253-300.

Shiao and Lin (1987) J Nat Prod 50:886-891.

Shibata et al., In: Economic and Medicinal Plant Research, Vol. 1 pp. 217-284. Academic Press, New York, 1985.

Soldati and Sticher (1980) Planta Med 39:348-357.

Stolketal., (1995). Diabetes 44: 11-15.

Sun et al., (1997). J. Nat. Prod. 60:203-206.

Sung et al., (200) Am J Chin Med. 28:205-16.

Taylor (1994). Diabetes 43:735-740.

Vuksan et al., (2000). Arch Int Med 160:1009- 1013.

Wakabayashi et al., (1998) Biochem Biophys Res Commun 246:725-730.

Wang et al., (1999). Endocronol 140:2117-2124.

Warram et al., (1990). Ann Inten Med 13:909-915.

Yki-Jarvinen et al., (1992). Endocrine Rev 13:415-431.

Yokozawa and Oura (1991). J Ethnopharmacol 34:79-82.

Yokozawa et al., (1985). Chem Pharm Bull 33:869-872.

Yokozawa et al., (1991). J Pharm Pharmacol 43:290-291.

Yokozawa et al., (1996). J Pharm Pharmacol 48:763-767.

Yuan et al., (1998a). Journal of Ethnopharmacology. 62(3):215-222.

Yuan et al., (1998b). American Journal of Chinese Medicine.26(1):47-55.

Yuan et al., (1999a). American Journal of Chinese Medicine.27(3-4):331-338.

Yuan et al., (1999b). Pain 83(3):631-635.

Yuan et al., (1999c). Amer. J. Physiol. 277:626-630.

Yuan et al., (2000). JAMA 283:367-372.

What is claimed is:

1. A method of treating diabetes comprising administering to an animal having diabetes an active compound from a berry from a plant of the Panax genus.

2. The method of claim 1, wherein the berry is from the ginseng species Panax ginseng or Panax quinquefolius.

3. The method of claim 2, wherein the active compound comprises an anti-hyperglycemic constituent.

4. The method of claim 3, wherein the active compound comprises a ginsenoside.

5. The method of claim 4, wherein the ginsenoside is Rg1, Re, Rb1, Rc, Rb2 or Rd.

6. The method of claim 5, wherein the ginsenoside is Re.

7. The method of claim 3, wherein the active compound comprises at least two ginsenosides.

8. The method of claim 3, wherein the active compound comprises non-ginsenoside components of berry extract.

9. The method of claim 3, wherein the active compound is ginsenoside free.

10. The method of claim 3, wherein the active compound comprises an anti-obesity constituent.

11. The method of claim 1, wherein the animal has non-insulin dependent diabetes.

12. The method of claim 1, wherein the animal is a mammal.

13. The method of claim 12, wherein the mammal is a human.

14. The method of claim 13, wherein the human is obese.

15. The method of claim 1, wherein the composition is administered via a parenteral route.

16. The method of claim 15, wherein the parenteral route is intraperitoneal, intravenous, subcutaneous, intramuscular, intradermal or transdermal.

17. The method of claim 1, wherein the composition is administered via an alimentary route.

18. The method of claim 17, wherein the alimentary route is oral, rectal, sublingual or buccal.

19. The method of claim 1, wherein the composition is administered as a dose.

20. The method of claim 19, wherein the dose is administered at least once a day.

21. The method of claim 19, wherein the dose is administered preprandial.

22. The method of claim 1, wherein the composition is administered as a series of doses.

23. A pharmaceutical composition comprising an active compound from a berry from a plant of the Panax genus and a pharmaceutically acceptable carrier.

24. The composition of claim 23, wherein the berry is from the ginseng species Panax ginseng or Panax quinquefolius.

25. The composition of claim 23, wherein the active compound comprises a ginsenoside.

26. The composition of claim 25, wherein the ginsenoside is Re.

27. A method of increasing body weight loss comprising administering to the animal an active compound from a berry from a plant of the Panax genus.

28. The method of claim 27, wherein the active compound comprises a ginsenoside.

29. The method of claim 28, wherein the ginsenoside is Re.

30. A method of treating an animal having hyperglycemia comprising administering to the animal an active compound from a berry from a plant of the Panax genus.

31. The method of claim 30, wherein the active compound comprises a ginsenoside.

32. The method of claim 31, wherein the ginsenoside is Re.

33. The method of claim 30, wherein the active compound comprises an anti-obesity constituent.

34. A method of treating an animal to decrease blood glucose levels comprising administering to the animal an active compound from a berry from a plant of the Panax genus.

35. The method of claim 34, wherein the active compound comprises an anti-hyperglycemic constituent.

36. The method of claim 35, wherein the active compound comprises a ginsenoside.

37. The method of claim 36, wherein the ginsenoside is Re.

38. The method of claim 35, wherein the active compound comprises an anti-obesity constituent.

39. A method of treating an animal to decrease plasma cholesterol comprising administering to the animal an active compound from a berry from a plant of the Panax genus.

40. The method of claim 39, wherein the active compound comprises a ginsenoside.

41. The method of claim 40, wherein the ginsenoside is Re.

42. A method of screening for an active compound from a berry of a plant of the Panax genus comprising:
a) obtaining berry extract; and
b) analyzing the extract for the active compound.

43. The method of claim 42, further comprising isolating the active compound.

44. The method of claim 43, further comprising identifying the isolated compound.

45. The method of claim 44, further comprising synthesizing the identified compound.

46. The method of claim 42, wherein the extract comprises at least one ginsenoside.

47. The method of claim 42, wherein the extract comprises non-ginsenoside components.

48. The method of claim 47, wherein the extract is ginsenoside free.

49. The method of claim 42, further comprising administering the fractions to an animal having hyperglycemia.

50. The method of claim 49, further comprising measuring blood glucose, wherein a decrease in blood glucose indicates that the fraction comprises an active compound.
